SEQUENCE LISTING (10 sequences)

SEQ ID NO: 1 - RNA, 97 nt, Homo sapiens
cugcuccuucucccauacccauugcauaucggaguugugaauucucaaaacaccuccugugugcauggauuacaggagggugagccuugucaucgug

SEQ ID NO: 2 - RNA, 22 nt, Homo sapiens
uacccauugcauaucggaguug

SEQ ID NO: 3 - RNA, 21 nt, Homo sapiens
accuccugugugcauggauua

SEQ ID NO: 4 - RNA, 7 nt, Homo sapiens
acccauu

SEQ ID NO: 5 - DNA, 7 bp, Homo sapiens
acccatt

SEQ ID NO: 6 - RNA, 7 nt, Homo sapiens
aaugggu

SEQ ID NO: 7 - RNA, 23 nt, Homo sapiens
cacaaaaccacuuuuaaugggua

SEQ ID NO: 8 - RNA, 23 nt, Homo sapiens
cacaaaaccacuuuuaccaaaua

SEQ ID NO: 9 - DNA, 35 bp, Homo sapiens
caaaaccacttttaccaaatacagagttaaatttg

SEQ ID NO: 10 - DNA, 35 bp, Homo sapiens
caaatttaactctgtatttggtaaaagtggttttg

(19) United States
(12) Patent Application Publication
(10) Pub. No.: US 2016/0108405 A1
(43) Pub. Date: Apr. 21, 2016

(54) LUNG CANCER DIAGNOSTICS AND THERAPEUTICS WITH MIR-660

(71) Applicant: BioMirna Holdings LTD, Sandymount (IE)

(72) Inventors: Gabriella Sozzi, Milan (IT); Ugo Pastorino, Milan (IT); Mattia Boeri, Milan (IT)

(21) Appl. No.: 14/885,536

(22) Filed: Oct. 16, 2015

Related U.S. Application Data
(60) Provisional application No. 62/065,217, filed on Oct. 17, 2014.

(51) Int. Cl. C12N 15/113; C12Q 1/68; G01N 33/574; C12N 15/86

(52) U.S. Cl. CPC C12N 15/1135; C12N 15/86; C12Q 1/6886; G01N 33/57423; C12N 2310/141; C12N 2750/14143; C12Q 2600/106; C12Q 2600/158; C12Q 2600/178

(57) ABSTRACT

Provided are methods of treating lung cancer in a patient in need thereof. The method includes administration to the patient a composition comprising a therapeutically effective amount of a compound that reduces the expression level of E3 ubiquitin-protein ligase MDM2. The compound in certain instances is a miR-660 miRNA, or a functional variant thereof. The patient in need of treatment in certain instances expresses miR-660 in a lung tissue sample or biological fluid sample at a level lower as compared to a control level derived from a subject or plurality of subjects that do not have lung cancer, or as compared to a control level derived from a lung cancer patient or plurality thereof, that have been given a favorable prognosis; expresses MDM2 at a higher level in a lung tissue sample or biological fluid sample as compared to a control level derived from a subject or plurality of subjects that do not have lung cancer, or as compared to a control level derived from a lung cancer patient or plurality thereof that have been given a favorable prognosis; and/or expresses p53 in a lung tissue sample or a biological fluid sample below a control level derived from a lung tumor tissue sample, or plurality thereof, or a biological fluid sample, or plurality thereof, obtained from a patient that has a favorable lung cancer prognosis; or a control level derived from a healthy subject.

### CROSS-REFERENCE TO RELATED APPLICATIONS

This application claims priority to and benefit of provisional application U.S. Ser. No. 62/065,217 filed on Oct. 17, 2014, the contents of which are incorporated herein by reference in their entirety.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is GENS-009-001US_SEQ.txt. The text file is 1.76 KB, was created on Oct. 16, 2015, and is being submitted electronically via EFS-Web.

### FIELD OF THE INVENTION

The present invention generally relates to lung cancer diagnostics and therapeutics. More specifically, the present invention relates to methods for treating lung cancer and methods for selecting patients who can benefit from the treatment methods described herein. In some embodiments, the invention is directed to the use of miR-660 in diagnostics, prognostics and treatment of lung cancer.

### BACKGROUND OF THE INVENTION

Lung cancer is the leading cause of cancer death worldwide, resulting in more than 1.4 million deaths per year (Jemal et al. (2011). CA Cancer J Clin 61, pp. 69-90). Non-molecular lung cancer diagnostics rely on radiological findings and histological analysis of biopsy tissue. Chest radiography (x-ray) can be used as a screening tool, albeit its low sensitivity and specificity. Computed tomography (CT) is much more sensitive for detecting small nodules in the lungs that are likely to represent earlier stages of lung cancer. CT screening trials have shown that chest radiographs miss 60% to 80% of the lung cancers detected by CT, but CT is more costly and delivers higher amounts of radiation to the patient. There is also a greater risk of over diagnosis, not only of nonmalignant lung nodules, but other incidental findings as well.

Lung cancer staging is determined using several criteria. The vast majority of lung cancers are carcinomas, i.e., malignancies that arise from epithelial cells. Based on histological criteria, there are two main types of lung carcinoma, categorized by the size and appearance of the malignant cells: non-small cell (about 85% of lung cancers) and small-cell lung carcinoma (about 15%). The non-small cell lung carcinomas (NSCLC) are grouped together because their prognosis and management are similar. There are three main sub-types: squamous cell lung carcinoma (25%), adenocarcinoma (60%), and large cell lung carcinoma (5%).

Non-small cell lung carcinoma is staged from IA (“one A”; early stage, typically associated with more favorable prognosis) to IV (“four”; advanced stage, typically associated with poor prognosis). Overall, lung cancer is staged based on the extent and size of the tumor (T), lymph nodes (N) involved, and presence of metastases (M).

Often, tumors are discovered as locally advanced or as metastatic disease, and despite improvements in molecular diagnosis and targeted therapies, the overall 5-year survival rate remains in the 10-20% range. Indeed, non-small cell lung cancer (NSCLC) is poorly chemosensitive to most of the available agents with response rates ranging from 10% to 25% (Ettinger et al. (2012). J. Natl Compr Canc Netw 8, pp. 740-801). The discovery of recurrent mutations in the epidermal growth factor receptor (EGFR) kinase (Lynch et al. (2004). N Engl J Med 350, pp. 2129-2139), as well as gene fusion products involving the anaplastic lymphoma kinase (ALK) (Soda et al., (2007). Nature 448, pp. 561-566), has led to a marked change in the treatment of patients with lung adenocarcinoma, the most common type of lung cancer (Youlden et al. (2008). J Thorac Oncol 3, pp. 819-831; Jackman and Johnson (2005). Lancet 366, pp. 1385-1396). To date, approximately 10% of lung cancers display mutations in the EGFR gene, the target for EGFR tyrosine kinase inhibitors (TKIs), while only about 5% of tumors have ALK rearrangements that can be targeted by ALK inhibitors (Herbst et al. (2008). N Engl J Med 359, pp. 1367-1380). Thus, the majority of lung tumors lack effective treatment and novel therapeutic strategies are still needed.

MicroRNAs (miRNAs) are short non-coding RNAs, 20-24 nucleotides long, that play important roles in almost all biological pathways (Bartel (2004). Cell, 116, pp. 281-297; Bartel (2009). Cell 136, pp. 215-233; Lewis et al. (2005). Cell 120, pp. 15-20; Lagos-Quintana et al. (2001) Science 294, pp. 853-858) and influence numerous cancer-relevant processes such as proliferation (Xiao et al. (2008) Nat Immunol 9, pp. 405-414), cell cycle (He et al., (2007). Nature 447, pp. 1130-1134), apoptosis (Cimmino et al., (2005) Proc Natl Acad Sci USA 102, pp. 13944-13949) and migration (Ma et al. (2007) Nature 449, pp. 682-688). MiRNAs are aberrantly expressed in different cancers (Iorio et al., 2005; Calin et al., 2005; Iorio and Croce, 2012; U.S. Patent Publication 2011/0251098) and contribute to carcinogenesis by promoting the expression of oncogenes or by inhibiting the expression of tumor suppressor genes (Croce, 2009). Many studies have demonstrated the critical role of miRNAs in lung cancer pathogenesis and their potential as biomarkers for lung cancer risk stratification (Raponi et al. (2009). Cancer Res 69, pp. 5776-5783), outcome prediction (Yanaihara et al. (2006). Cancer Cell 9, pp. 189-198) and classification of histological subtypes (Takamizawa et al. (2004). Cancer Res 64, pp. 3753-3756; Bishop et al. (2010). Clin Cancer Res 16, pp. 610-619). MiRNAs released by cells can also be found in biological fluids such as plasma, serum, and urine (U.S. Pat. No. 8,486,626) making them suitable as biomarkers in lung cancers such as NSCLC (Boeri et al. (2011) Proc Natl Acad Sci USA 108, pp. 3713-3718; Sozzi et al. (2014) J Clin Oncol 32, pp. 768-773; U.S. Pat. No. 8,735,074; U.S. Patent Application Publication 2012/0329060).

MiR-660 has been reported to be up-regulated in chronic lymphocytic leukemia (Zhu et al. (2012) Carciogenesis 33, pp. 1294-1301; Ferrer et al. (2013) Leuk Lymphoma 54, pp. 2016-2022) and also in leukemic cells after treatment with 4-hydroxynonenal, a compound that induces differentiation and blocks proliferation of leukemic cells (Pizzimenti et al. (2009). Free Radic Biol Med 46, pp. 282-288). Furthermore, miR-660 up-regulation was observed during in vitro differentiation of myoblast (Dmitriev et al., 2013a) and facioscapulohumeral muscular dystrophy (Dmitriev et al., 2013b). MiR-660 is also involved in the expansion and production of platelets during megakaryopoiesis (Emmrich et al., 2012). MiR-660 was shown to be de-regulated in plasma samples of NSCLC patients identified in a low-dose computed tomography (LDCT) screening trial (Boeri et al., 2011). Despite some evidence of miR-660 de-regulation in cancer, little is known about its role in lung tumorigenesis and its putative target genes.

The p53 tumor suppressor protein is a key regulator of cell cycle G0/G1 checkpoint, senescence and apoptosis in response to cellular stress signals (Levine (1997). Cell 88, pp. 323-331; Wu and Levine (1997). Mol Med 3, pp. 441-451). Mouse double minute 2 (MDM2), a p53 E3 ubiquitin ligase (Honda et al. (1997). FEBS Lett 420, pp. 25-27), is the principal negative regulator of the level and function of p53 (Montes de Oca Luna et al. (1995). Nature 378, pp. 203-206; Chen et al. (1996). Mol Cell Biol 16, pp. 2445-2452). MDM2 regulates p53 by various mechanisms (Kubbutat et al. (1997). Nature 387, pp. 299-303; Moll and Petrenko (2003). Mol Cancer Res 1, pp. 1001-1008), e.g., by binding transactivation region of p53 (Kussie et al. (1996). Science 274, pp. 948-953; Momaid et al. (1992). Cell 69, pp. 1237-1245), promoting nuclear export and cytoplasmic accumulation of p53 by monoubiquitination (Haupt et al. (1997) Nature 387, pp. 296-299; Lai et al. (2001). J Biol Chem 276, pp. 31357-31367) and inducing p53 proteosomal degradation by polyubiquitination (Feng et al. (2004). J Biol Chem 279, pp. 35510-35517). In addition, the MDM2 gene is amplified or overexpressed in a variety of human cancers, such as sarcoma (Oliner et al. (1992). Nature 358, pp. 80-83), lymphoma (Capoulade et al. (1998) Oncogene 16, pp. 1603-1610), breast cancer (Marchetti et al. (1995). J Pathol 175, pp. 31-38, lung cancer (Marchetti et al. (1995). Diagn Mol Pathol 4, pp. 93-97) and testicular germ cell tumor (Riou et al. (1995). Mol Carcinog 12, pp. 124-131), and expression of p53 in lung tumor samples is correlated with a positive prognosis (Xu et al., 2013). Additionally, NSCLC patients that have an MDM2 variant that is associated with p53 overexpression have better prognosis than patients with an MDM2 variant that is associated with less p53 overexpression (Han et al., 2008). Several miRNAs target MDM2, including the miR-143/miR-145 cluster which can be induced by p53 (Zhang et al. (2013). Oncogene 32, pp. 61-69) as well as miR-25 and miR-32, which inhibit tumor glioblastoma growth in mouse brain (Suh et al. (2014). Proc Natl Acad Sci USA 109, pp. 5316-5321).

### SUMMARY OF THE INVENTION

In one aspect, the present invention relates to a pharmaceutical composition comprising a compound that reduces the expression level of E3 ubiquitin-protein ligase MDM2. The expression level in one embodiment, is protein expression. In another embodiment, the MDM2 expression is mRNA expression. In one embodiment, the pharmaceutical composition comprises an RNA interference (RNAi) compound that targets MDM2 mRNA expression, for example, a small interfering RNA (siRNA), short hairpin RNA (shRNA) or a micro RNA (miRNA). In a further embodiment, the pharmaceutical composition comprises a miR-660 oligonucleotide (e.g., a miRNA of SEQ ID NO:2 or 3, or a functional variant thereof).

In some embodiments of the pharmaceutical composition comprising a compound that reduces the expression level of E3 ubiquitin-protein ligase MDM2 further comprises a pharmaceutically acceptable carrier.

As provided above, in one embodiment described herein, a pharmaceutical composition comprising an RNAi compound that targets MDM2 mRNA expression is provided. In one embodiment, the compound is a miR, e.g., miR-660 (SEQ ID NO: 2, 3) or a miR-660 pre-miR (SEQ ID NO: 1), or a functional variant thereof. In one embodiment, the miR is at least 90% identical to SEQ ID NO: 2 or SEQ ID NO: 3. In one embodiment, the compound is a miR-660 functional variant and comprises one or more modified nucleotides. For example, the compound in one embodiment is a miR-660 functional variant comprising at least one, at least two or at least three nucleotides and is stable in the patient for a longer period of time than the compound of SEQ ID NO:2 or SEQ ID NO: 3. In one embodiment, the compound is encoded by a vector (e.g., a viral vector such as an adeno-associated virus (AAV) vector, or a plasmid based expression vector).

In yet another embodiment, the compound that reduces MDM2 mRNA expression is an RNA compound that comprises the sequence ACCCAUU (SEQ ID NO: 4) or ACCCATT (SEQ ID NO: 5). In a further embodiment, the compound comprising the sequence SEQ ID NO: 4 or SEQ ID NO: 5 is an siRNA, a shRNA, a miRNA or an antisense inhibitor.

In one embodiment, the compound that reduces MDM2 mRNA expression is encoded by a vector. In a further embodiment, the vector encodes a sequence selected from SEQ ID NO: 1, 2, 3, or 4, or a variant thereof.

In another aspect, the present invention relates to a method of treating cancer for example, lung cancer. In one embodiment, the method comprises administering to a patient in need thereof, a composition comprising an effective amount of one of the compounds that reduces MDM2 expression. In a further embodiment, the cancer is lung cancer. The lung cancer is a small cell lung cancer or a non-small cell lung cancer (NSCLC). The compound, as described in embodiments herein, is an RNAi compound that targets MDM2 expression, e.g., a miR-660 miRNA or a variant thereof. In one embodiment, the patient in need of treatment (i) expresses miR-660 in a lung tissue sample or biological fluid sample at a level lower than a control level derived from a subject or plurality of subjects that do not have lung cancer, or as compared to a control level derived from a lung cancer patient or plurality thereof, that have been given a favorable prognosis; (ii) expresses MDM2 at a higher level in a lung tissue sample or biological fluid sample as compared to a control level derived from a subject or plurality of subjects that do not have lung cancer, or as compared to a control level derived from a lung cancer patient or plurality thereof that have been given a favorable prognosis; and/or (iii) expresses p53 in a lung tissue sample or a biological fluid sample below a control level derived from a lung tumor tissue sample (or plurality thereof) or a biological fluid sample (or plurality thereof) obtained from a patient that has a favorable lung cancer prognosis; or a control level derived from a healthy subject.

In yet another aspect of the invention, a patient in need of treatment is selected for treatment via a miRNA blood test. For example, a patient's blood sample is interrogated for the expression of at least five, at least 10, at least 15, at least 20 or 24 of the miRNAs set forth in Table A. Based on ratios of expression ratios of miRNA pairs, a patient is either selected or not selected for therapy with one of the compositions described herein. For example, if the ratio of the miRNA pair exceeds a cut-off value determined from a comparison to a control sample or a plurality thereof, the ratio is assigned a positive score. In one embodiment, if at least nine of the miRNA expression ratios are assigned a positive score, the patient is selected for therapy. In another embodiment. In one embodiment, the miRNA pairs comprise 106a/140-5p, 106a/142-3p, 126/140-5p, 126/142-3p, 133a/142-3p, 140-5p/17, 142-3p/148a, 142-3p/15b, 142-3p/17, 142-3p/21, 142-3p/221, 142-3p/30b, and 320/660, or the inverse ratios thereof. In a further embodiment, the miRNA pairs comprise 106a/660, 106a/92a, 126/660, 140-5p/197, 140-5p/28-3p, 142-3p/145, 142-3p/197, 142-3p/28-3p, 17/660, 17/92a, 197/660, 197/92a, 19b/660, or 28-3p/660, or the inverse ratios thereof.

In yet another embodiment, the miRNA pairs comprise 106a/660, 106a/92a, 126/660, 140-5p/197, 140-5p/28-3p, 142-3p/145, 142-3p/197, 142-3p/28-3p, 17/660, 17/92a, 197/660, 197/92a, 19b/660, and 28-3p/660, or the inverse ratios thereof.

TABLE A

| | | |
|---|---|---|
| hsa-miR-16 | hsa-miR-320 | hsa-miR-148a |
| hsa -miR-17 | hsa -miR-451 | hsa -miR-15b |
| hsa-miR-21 | hsa-miR-660 | hsa-miR-19b |
| hsa -miR-101 | hsa -miR-106a | hsa -miR-28-3p |
| hsa-miR-126 | hsa-miR-133a | hsa-miR-30b |
| hsa -miR-145 | hsa -miR-140-3p | hsa -miR-30c |
| hsa-miR-197 | hsa-miR-140-5p | hsa-miR-486-5p |
| hsa -miR-221 | hsa -miR-142-3p | hsa -miR-92a |

In some embodiments, a patient in need of treatment with the compositions and methods provided herein has a lung tumor tissue sample or a biological fluid sample that expresses p53. In a further embodiment, p53 is expressed below a level of p53 expression in a lung tumor tissue sample or a biological fluid sample obtained from a patient that has a favorable lung cancer prognosis.

In one embodiment, a sample (e.g., biological fluid sample such as a blood sample) from the patient expresses miR-660 (i) below a miR-660 level in a noncancerous lung tissue sample from the patient, (ii) below a miR-660 level in a subject that does not have lung cancer (e.g., a healthy subject) or (iii) below a mean miR-660 level in a plurality of subjects that do not have lung cancer.

In one embodiment, a miR-660 level in a plasma sample from the patient in need of treatment is below a miR-660 level in a plasma sample from a subject that does not have lung cancer (e.g., a healthy subject) or the average miR-660 plasma level in a plurality of subjects that do not have lung cancer.

In some embodiments, a lung tumor tissue sample from the patient in need of treatment expresses MDM2 (e.g., MDM2 protein or mRNA). In some embodiments, the MDM2 expression level in the lung tumor tissue is greater than an MDM2 level in a noncancerous lung tissue sample from the patient in need of treatment or greater than an MDM2 level in a subject that does not have lung cancer (e.g., a healthy subject).

In some embodiments, the patient in need of treatment is selected for treatment by one of the methods disclosed in U.S. Patent Application Publication No. US 2015/0191794 published Jul. 9, 2015), the contents of which are incorporated by reference herein in their entireties for all purposes.

Another aspect of the invention relates to a method of evaluating the prognosis of a lung cancer patient. The method comprises measuring the concentration of miR-660 in a tissue sample or bodily fluid of the patient, wherein a miR-660 concentration in the patient below a miR-660 concentration in the same fluid or tissue of a plurality of lung cancer patients that had a favorable prognosis indicates that the patient has a poor prognosis.

In yet another aspect, another method of diagnosing lung cancer in a patient is provided. The method comprises measuring the concentration of mir-660 in (a) tissue suspected of being lung cancer in the patient and (b) normal tissue of the patient, wherein a lower level of miR-660 in the suspected tissue than in the normal tissue indicates that the suspected tissue is lung cancer.

### BRIEF DESCRIPTION OF THE DRAWINGS

FIGS. 1A-1C are graphs showing miR-660 efficient expression manipulation in human lung cancer lines. The graphs on the left show the relative expression of miR-660 after transient transfection with miR-660 mimic or control at 24 h, 48 h, 72 h or 96 h; the graphs on the right show the relative expression of mir-660 after stable transfection with mir-660 or control lentiviral vector at 10 d and 30 d. These determinations were made in three different cell lines: H460 (FIG. 1A), A549 (FIG. 1B) and H1299 (FIG. 1C). All data are expressed as mean±standard error of the mean (SEM). (n=3, *p<0.05 vs. miRNA mimic control (“mim-ctr”)).

FIGS. 2A-2B are graphs showing that miR-660 is down-regulated in tumor tissue and plasma. FIG. 2A is a dot plot showing miR-660 levels in plasma samples. Data were normalized on the average of each card. *p<0.05 vs each group. FIG. 2B is a histogram showing miR-660 expression levels in lung cancers compared to normal tissues. Data are expressed as mean±standard error of the mean (SEM). *p<0.05 vs. normal tissues.

FIGS. 3A-3D are images and graphs showing antitumoral effects of miR-660 (SEQ ID NO: 2). FIG. 3A shows that miR-660 decreases migratory capacity of lung cancer cells in a Transwell® assay (n=5), and FIG. 3B shows that miR-660 decreases invasive capacity of lung cancer cells in a Transwell® assay (n=5). Representative images of migrated/invaded cells for each condition are shown. Migration and invasion data are expressed as the number of migrated mir-660 over-expressing cells vs. the number of migrated control cells. All data are expressed as mean±standard error of the mean (SEM). *p<0.05 vs. cells transfected with control. FIG. 3C shows the proliferation of cells transfected with miR-660 or control. Viable cells were counted with trypan blue at 24, 72 and 120 hours to measure cell growth. Graphs show the proliferation reduction of miR-660 over-expressing cells compared to control cells (n=5). FIG. 3D shows apoptosis, measured as annexin V^{pos}/PI^{neg} cells and expressed as a fold increase compared to cell transfected with mimic control (n=5). All data are expressed as mean±SEM. *p<0.05 vs. cells transfected with control.

FIGS. 4A-4B are graphs showing that mir-660 over-expression reduces lung cancer cell growth. FIG. 4A shows cells that were transfected with mir-660 or control and viable cells were counted with trypan blue at 72 and 120 hours to measure cell growth. Graphs show cell proliferation of mir-660 over-expressing cells compared to control cells (n=5). FIG. 4B shows apoptosis measured by flow cytometry as annexin V^{pos}/PI^{neg} cells (left panel) and graphs show the number of apoptotic cells compared to cell transfected with mimic control (right panel) (n=5). All data are expressed as mean±SEM. *p<0.05 vs. cells transfected with control.

FIGS. 5A-5B are a graphic representation and a graph showing that MDM2 is a direct target of miR-660. FIG. 5A shows a predicted MDM2 3′UTR-binding site for miR-660. The figure shows alignment of a miR-660 sequence (SEQ ID NO: 2) with a portion of the wild type MDM2 3′UTR (SEQ ID NO: 7) and mutated MDM2 (SEQ ID NO: 8). FIG. 5B is a bar graph showing average luciferase activity. Reporter systems were transfected in HEK293 with wild type MDM2, mutated MDM2, or EMPTY 3′UTR, in combination with miR-660 mimics or control. All data are expressed as mean±SEM. (n=5; *p<0.05).

FIGS. 6A-6B are graphs and photographs of western blots showing that MDM2 expression is down-modulated after miR-660 over-expression. FIG. 6A is a graph showing MDM2 mRNA levels in lung cancer cells transfected with mimic miR-660 or mimic control (n=5). FIG. 6B shows results of MDM2 analysis by western blot (n=4) and representative western blot bands. All data are expressed as mean±SEM. (*p<0.05).

FIGS. 7A-7C are graphs and photographs showing that mir-660 increased p53 levels and function. FIG. 7A shows p53 levels after mir-660 over-expression measured by ELISA (n=4). FIG. 7B shows p21 mRNA levels in lung cancer cells transfected with mimic mir-660 or mimic control (n=4). FIG. 7C shows p21 expression analysis by western blot (n=4) and representative western blot bands for all cell lines. All data are expressed as mean±SEM. (*p<0.05).

FIGS. 8A-8F are graphs and photographs showing that stable mir-660 expression reduced p53 wt cancer cell functionality. FIG. 8A shows that stable mir-660 over-expression decreases migratory capacity of lung cancer cells in a Transwell® assay (n=3). FIG. 8B shows that stable mir-660 over-expression decreases invasive capacity of lung cancer cells in Transwell® assay (n=3). FIG. 8C shows viable cells that were counted with trypan blue at 72 and 120 hours to measure cell growth. Graphs show the proliferation reduction of mir-660 over-expressing cells compared to control cells (n=3). FIG. 8D shows apoptosis measured by flow cytometry as annexin V^{pos}/PI^{neg} cells and expressed as number of apoptotic cells compared to control (n=3). FIG. 8E shows representative graphs of cell cycle analysis in stable mir-660 over-expressing cells compared to controls. FIG. 8F shows the results of MDM2 analysis by western blot (n=3) and representative western blot bands. All data are expressed as mean±SEM. *p<0.05 vs. mir-660 cells with control.

FIGS. 9A-9C are graphs and photographs showing that mir-660 inhibited xenograft tumor growth in mice. Graphs show tumor growth of mir-660 over-expressing cells subcutaneously (s.c.) injected in both flanks of nude mice compared to control (n=5 per group). MiRNAs were stable transfected in (FIG. 9A) NCI-H460, (FIG. 9B) A549 and (FIG. 9C) H1299. All data are expressed as mean±SEM. (*p<0.05 vs. mim-ctr). Representative images of tumor size for each condition (right panels).

FIGS. 10A-10C are graphs showing inhibition of xenograft tumor growth in mice with miR-660. Graphs show tumor growth of miR-660 over-expressing cells s.c injected in both flanks of nude mice compared to control (n=5 per group). MiRNAs were transiently (left panels) or stably transfected (right panels) in (FIG. 10A) NCI-H460, (FIG. 10B) A549 and (FIG. 10C) H1299 cells. All data are expressed as mean±SEM. (*P<0.05 vs mim-ctr).

FIGS. 11A-11D are graphs showing transient mir-660 over-expression delay tumor growth in mice. Graphs show tumor growth of mir-660 over-expressing cells s.c. injected in both flanks of nude mice compared to control (n=5 per group). MiRNAs were transiently transfected in (FIG. 11A) NCI-H460, (FIG. 11B) A549, and (FIG. 11C) H1299. (FIG. 11D) Relative expression of mir-660 after transient transfection with mir-660 mimic or control in mice tumors. All data are expressed as mean±SEM. (*p<0.05 vs. mim-ctr).

### DETAILED DESCRIPTION OF THE INVENTION

Unless defined otherwise all technical and scientific terms used herein have the same meaning as commonly understood to one of ordinary skill in the art to which this disclosure belongs.

As used herein, the singular forms “a”, “an” and “the” are intended to include the plural forms as well, unless the context clearly indicates otherwise. Additionally, the use of “or” is intended to include “and/or”, unless the context clearly indicates otherwise.

As used herein, a “gene” is a polynucleotide that encodes a discrete product, whether RNA or proteinaceous in nature. It is appreciated that more than one polynucleotide may be capable of encoding a discrete product. The term includes alleles and polymorphisms of a gene that encodes the same product, or a functionally associated (including gain, loss, or modulation of function) analog thereof, based upon chromosomal location and ability to recombine during normal mitosis.

A “sequence” or “gene sequence” as used herein is a nucleic acid molecule or polynucleotide composed of a discrete order of nucleotide bases. The term includes the ordering of bases that encodes a discrete product (i.e., “coding region”), whether RNA or proteinaceous in nature. It is appreciated that more than one polynucleotide may be capable of encoding a discrete product. It is also appreciated that alleles and polymorphisms of the human gene sequences may exist and may be used in the practice of the disclosure to identify the expression level(s) of the gene sequences or an allele or polymorphism thereof. Identification of an allele or polymorphism depends in part upon chromosomal location and ability to recombine during mitosis.

An “expressed sequence” is a sequence that is transcribed by cellular processes within a cell. To detect an expressed sequence, a region of the sequence that is unique relative to other expressed sequences may be used. An expressed sequence may encode a polypeptide product or not be known to encode any product. So an expressed sequence may contain open reading frames or no open reading frames. Non-limiting examples include regions of about 8 or more, about 10 or more, about 12 or more, about 14 or more, about 16 or more, about 18 or more, about 20 or more, about 22 or more, about 24 or more, about 26 or more, about 28 or more, or about 30 or more contiguous nucleotides within an expressed sequence may be used. The term “about” as used in the previous sentence refers to an increase or decrease of 1 from the stated numerical value. The physical form of an expressed sequence may be an RNA molecule or the corresponding cDNA molecule.

The terms “correlate” or “correlation” or equivalents thereof refer to an association between expression of one or more genes and another event, such as, but not limited to, physiological phenotype or characteristic, such as tumor type.

A “polynucleotide” is a polymeric form of nucleotides of any length, either ribonucleotides or deoxyribonucleotides. This term refers only to the primary structure of the molecule. Thus, this term includes double- and single-stranded DNA and RNA. It also includes known types of modifications including labels known in the art, methylation, “caps”, substitution of one or more of the naturally occurring nucleotides with an analog, and internucleotide modifications such as uncharged linkages (e.g., phosphorothioates, phosphorodithioates, etc.), as well as unmodified forms of the polynucleotide.

The term “amplify” is used in the broad sense to mean creating an amplification product can be made enzymatically with DNA or RNA polymerases. “Amplification” as used herein, generally refers to the process of producing multiple copies of a desired sequence, particularly those of a sample. “Multiple copies” mean at least 2 copies. A “copy” does not necessarily mean perfect sequence complementarity or identity to the template sequence. Methods for amplifying mRNA are generally known in the art, and include reverse transcription PCR (RT-PCR) and quantitative PCR (or Q-PCR) or real time PCR. Alternatively, RNA may be directly labeled as the corresponding cDNA by methods known in the art.

By “corresponding”, it is meant that a nucleic acid molecule shares a substantial amount of sequence identity with another nucleic acid molecule. Substantial amount means at least 95%, usually at least 98% and more usually at least 99%, and sequence identity is determined using the BLAST algorithm, as described in Altschul et al. (1990), J. Mol. Biol. 215, pp. 403-410, incorporated by reference herein in its entirety, e.g., by using the published default setting, i.e., parameters w=4, t=17.

The terms “label” or “labeled” refer to a composition, compound or moiety capable of producing a detectable signal indicative of the presence of the labeled molecule. Suitable labels include radioisotopes, nucleotide chromophores, enzymes, substrates, fluorescent molecules, chemiluminescent moieties, magnetic particles, bioluminescent moieties, and the like. As such, a label is any composition detectable by spectroscopic, photochemical, biochemical, immunochemical, electrical, optical, or chemical means.

The term “support” refers to conventional supports such as beads, particles, dipsticks, fibers, filters, membranes and silane or silicate supports such as glass slides.

“Expression” and “gene expression” include transcription and/or translation of nucleic acid material. Expression levels of an expressed sequence may optionally be normalized by reference or comparison to the expression level(s) of one or more control expressed genes. These “normalization genes” have expression levels that are relatively constant in all members of the plurality or group of known tumor types.

As used herein, the term “comprising” and its cognates are used in their inclusive sense; that is, equivalent to the term “including” and its corresponding cognates.

Conditions that “allow” an event to occur or conditions that are “suitable” for an event to occur, such as hybridization, strand extension, and the like, or “suitable” conditions are conditions that do not prevent such events from occurring. Thus, these conditions permit, enhance, facilitate, and/or are conducive to the event. Such conditions, known in the art and described herein, depend upon, for example, the nature of the nucleotide sequence, temperature, and buffer conditions. These conditions also depend on what event is desired, such as hybridization, cleavage, strand extension or transcription.

Sequence “mutation,” as used herein, refers to any sequence alteration in the sequence of a gene disclosed herein interest in comparison to a reference sequence. A sequence mutation includes single nucleotide changes, or alterations of more than one nucleotide in a sequence, due to mechanisms such as substitution, deletion, or insertion. Single nucleotide polymorphism (SNP) is also a sequence mutation as used herein. Because embodiments of the present invention are based in part on the relative level of gene expression, mutations in non-coding regions of genes as disclosed herein may also be assayed in the practice of the disclosure.

“Detection” or “detecting” includes any means of detecting, including direct and indirect determination of the level of gene expression and changes therein.

As used herein, the term “treat” is meant to describe a process by which a sign or symptom of a disorder is reduced in severity or eliminated. Alternatively, or in addition, a disorder which can occur in multiple locations, is treated if that disorder is eliminated within at least one of multiple locations.

Effective dosages are expected to decrease the severity of a sign or symptom. For instance, a sign or symptom of a disorder, which can occur in multiple locations, is alleviated if the severity of the disorder is decreased within at least one of multiple locations.

As used herein, the term “severity” is meant to describe an unfavorable prognosis for a subject, a progression of a disorder to a more deleterious stage, a presentation of a sign or symptom or a diagnosis of an additional or secondary disorder, a requirement for invasive, experimental, or high-risk medical treatment, an indication that the disorder has become systemic rather than local or that the disorder has invaded additional or secondary bodily systems, the potential of a disorder to transform from a benign to malignant state, or the potential of a disorder to escalate from a state that is managed by preventative, daily, or routine medicine to a crises state that is managed by emergency medicine or specialize care centers.

As used herein, the term “severity” is also meant to describe the potential of cancer to transform from a precancerous, or benign, state into a malignant state. Alternatively, or in addition, severity is meant to describe, for instance, a cancer stage or grade. In additional aspects of the invention, severity describes the number and location of secondary cancers as well as the operability or drug-accessibility of those tumors. In these situations, prolonging the life expectancy of the subject and/or reducing pain, decreasing the proportion of cancerous cells or restricting cells to one system, and improving cancer stage/tumor grade/histological grade/nuclear grade are considered alleviating a sign or symptom of the cancer.

As used herein the term “symptom” is defined as an indication of disease, illness, or injury in the body. Symptoms are felt or noticed by the individual experiencing the symptom, but may not easily be noticed by others. Others are defined as non-health-care professionals.

As used herein the term “sign” is also defined as an indication of disease, illness, or injury in the body. Signs are defined as things that can be seen by a doctor, nurse, or other health care professional.

“miR-660” as used herein is any miRNA that is derived from the pre-miRNA having the stem-loop sequence 5′-CUGCUCCUUCUCCCAUACCCAUUGCAUAUCGGAGUUGUGAAUUCUCAAAACACCU CCUGUGUGCAUGGAUUACAGGAGGGUGAGCCUUGUCAUCGUG-3′ (SEQ ID NO: 1), or a functional variant thereof. For example, in one embodiment, “mir-660” refers to miR-660-5p, having the ribonucleotide sequence 5′-uacccauugcauaucggaguug-3′ (SEQ ID NO: 2). In another embodiment, the “mir-660” refers to miR-660-3p, having the ribonucleotide sequence 5′-accuccugugugcauggauua-3′ (SEQ ID NO: 3). In certain embodiments of the invention, at least one of the miR-660s is substituted with a modified nucleotide which does not substantially affect base pairing of miR-660 with other nucleic acids. SEQ ID NOs: 2 and 3 are shown underlined in the pre-miRNA below.

(SEQ ID NO: 1)
5′-CUGCUCCUUC UCCCAUACCC AUUGCAUAUC GGAGUUGUGA
AUUCUCAAAA CACCUCCUGU GUGCAUGGAU UACAGGAGGG
UGAGCCUUGU CAUCGUG-3′.

In some embodiments, the pre-miRNA of SEQ ID NO: 1 is processed into the underlined sequences by the RNase III enzyme Dicer in the cytoplasm. In some diagnostic or therapeutic embodiments, particularly where a Dicer is present, hsa-miR-660-5p or has-miR-660-3p (or functional variants thereof) can substitute for miR-660, since hsa-miR 660 is the therapeutic compound. The term “miR-660” also encompasses functional variants of wide-type miR-660, miR-660 mimics or functional variants thereof.

Thus, the term “microRNA mimic” refers to synthetic non-coding RNAs (i.e., the miRNA is not obtained by purification from a source of the endogenous miRNA) that are capable of entering the RNAi pathway and regulating gene expression. In some embodiments, the miR-660 mimic is hsa-miR-660-5p (SEQ ID NO: 2).

As used herein, the term “functional variant” of miR-660, refers to a nucleic acid that is at least 75% (e.g., at least 80%, at least 85%, at least 90%, at least 95%) identical in sequence to miR-660 and is capable of having one or more biological activities of miR-660. In one embodiment, the functional variant of miR-660 reduces the expression level of MDM2 mRNA. In some embodiments, a functional variant includes a non-natural nucleic acid, or a plurality thereof.

As used herein, the term “therapeutically effective amount” means the amount of a compound that, when administered to a patient for treating a state, disorder or condition, is sufficient to effect such treatment. The “therapeutically effective amount” will vary depending on the compound, the disease and its severity and the age, weight, physical condition and responsiveness of the mammal to be treated.

The present invention is based in part on the discovery that miR-660 interacts with MDM2 to reduce the MDM2-p53 interaction. The many applications of this newly discovered interaction include the diagnosis of lung cancer and other cancers, the prognosis of lung cancer and other cancers, and the treatment of lung cancer and other cancers.

In one aspect of the invention, the present invention provides a method of treating a cancer patient in need thereof, for example a lung cancer patient, and pharmaceutical compositions useful therefor. In one aspect, the therapeutic method comprises administering to the patient in need thereof a composition comprising a therapeutically effective amount of a compound that reduces the expression level of E3 ubiquitin-protein ligase MDM2, or reduces the interaction of MDM2 with p53 (e.g., an MDM2 antibody or fragment thereof). In some embodiments, the compound reduces the expression level of the MDM2 protein, MDM2 mRNA or a combination thereof. In one embodiment, the patient in need of treatment (i) expresses miR-660 in a lung tissue sample or biological fluid sample at a level lower than a control level derived from a subject or plurality of subjects that do not have lung cancer, or as compared to a control level derived from a lung cancer patient or plurality thereof, that have been given a favorable prognosis; (ii) expresses MDM2 at a higher level in a lung tissue sample or biological fluid sample as compared to a control level derived from a subject or plurality of subjects that do not have lung cancer, or as compared to a control level derived from a lung cancer patient or plurality thereof that have been given a favorable prognosis; and/or (iii) expresses p53 in a lung tissue sample or a biological fluid sample below a control level derived from a lung tumor tissue sample (or plurality thereof) or a biological fluid sample (or plurality thereof) obtained from a patient that has a favorable lung cancer prognosis; or a control level derived from a healthy subject.

In some embodiments, the compound that reduces the expression level of E3 ubiquitin-protein ligase MDM2 is a nucleic acid. In some embodiments, the compound is an RNA interfering agent, e.g., an siRNA molecule, an shRNA molecule or a miRNA, which reduces the expression level of MDM2. In yet another embodiment, the compound that reduces the interaction of MDM2 with p53 is an antibody that binds MDM2, or a fragment thereof.

As used herein, the term “RNA interfering agent” is intended to encompass those forms of gene silencing mediated by double-stranded RNA, regardless of whether the RNA interfering agent comprises an siRNA, miRNA, shRNA or other double-stranded RNA molecule.

“Short interfering RNA” (siRNA), also referred to herein as “small interfering RNA” is defined as an RNA agent which functions to inhibit expression of a target gene, e.g., by RNAi. An siRNA may be chemically synthesized, produced by in vitro transcription, or produced within a host cell. In one embodiment, siRNA is a double stranded RNA (dsRNA) molecule of about 15 to about 40 nucleotides in length, for example about 15 to about 28 nucleotides, about 19 to about 25 nucleotides in length, or about 19, about 20, about 21, about 22, or about 23 nucleotides in length, and may contain a 3′ and/or 5′ overhang on each strand having a length of about 0, 1, 2, 3, 4, or 5 nucleotides. The length of the overhang is independent between the two strands, i.e., the length of the overhang on one strand is not dependent on the length of the overhang on the second strand. In some embodiments, the siRNA is capable of promoting RNA interference through degradation or specific post-transcriptional gene silencing (PTGS) of the target messenger RNA (mRNA).

siRNAs also include small hairpin (also called stem loop) RNAs (shRNAs). In one embodiment, these shRNAs are composed of a short (e.g., about 19 to about 25 nucleotide) antisense strand, followed by a nucleotide loop of about 5 to about 9 nucleotides, and the analogous sense strand. Alternatively, the sense strand may precede the nucleotide loop structure and the antisense strand may follow. These shRNAs may be contained in plasmids, retroviruses, and lentiviruses and expressed from, for example, the pol III U6 promoter, or another promoter (see, e.g., Stewart, et al. (2003) RNA April; 9(4):493-501, incorporated by reference herein in its entirety). The target gene or sequence of the RNA interfering agent may be a cellular gene or genomic sequence, e.g. the MDM2 sequence. An siRNA may be substantially homologous to the target gene or genomic sequence, or a fragment thereof. As used in this context, the term “homologous” is defined as being substantially identical, sufficiently complementary, or similar to the target mRNA, or a fragment thereof, to effect RNA interference of the target. In addition to native RNA molecules, RNA suitable for inhibiting or interfering with the expression of a target sequence includes RNA derivatives and analogs. Preferably, the siRNA is identical to its target. The siRNA preferably targets only one sequence. Each of the RNA interfering agents, such as siRNAs, can be screened for potential off-target effects by, for example, expression profiling. Such methods are known to one skilled in the art and are described, for example, in Jackson et al. Nature Biotechnology 6:635-637, 2003, incorporated by reference herein in its entirety. In addition to expression profiling, one may also screen the potential target sequences for similar sequences in the sequence databases to identify potential sequences which may have off-target effects. For example, according to Jackson et al. (Nature Biotechnology 6:635-637, 2003), fifteen, or perhaps as few as eleven contiguous nucleotides, of sequence identity are sufficient to direct silencing of non-targeted transcripts. Therefore, one may initially screen the proposed siRNAs to avoid potential off-target silencing using the sequence identity analysis by any known sequence comparison methods, such as BLAST. siRNA sequences are chosen to maximize the uptake of the antisense (guide) strand of the siRNA into RISC and thereby maximize the ability of RISC to target human GGT mRNA for degradation. siRNA molecules need not be limited to those molecules containing only RNA, but, for example, further encompasses chemically modified nucleotides and non-nucleotides, and also include molecules wherein a ribose sugar molecule is substituted for another sugar molecule or a molecule which performs a similar function. Moreover, a non-natural linkage between nucleotide residues can be used, such as a phosphorothioate linkage. The RNA strand can be derivatized with a reactive functional group of a reporter group, such as a fluorophore. Particularly useful derivatives are modified at a terminus or termini of an RNA strand, typically the 3′ terminus of the sense strand. For example, the 2′-hydroxyl at the 3′ terminus can be readily and selectively derivatized with a variety of groups. Other useful RNA derivatives incorporate nucleotides having modified carbohydrate moieties, such as 2′O-alkylated residues or 2′-O-methyl ribosyl derivatives and 2′-O-fluoro ribosyl derivatives.

The RNA bases may also be modified in the RNAi compounds provided herein. Any modified base useful for inhibiting or interfering with the expression of a target sequence may be used. For example, halogenated bases, such as 5-bromouracil and 5-iodouracil can be incorporated. The bases may also be alkylated, for example, 7-methylguanosine can be incorporated in place of a guanosine residue. Non-natural bases that yield successful inhibition can also be incorporated. siRNA modifications amenable for use with the present invention include 2′-deoxy-2′-fluorouridine or locked nucleic acid (LNA) nucleotides and RNA duplexes containing either phosphodiester or varying numbers of phosphorothioate linkages. Such modifications are known to one skilled in the art and are described, for example, in Braasch et al., Biochemistry, 42: 7967-7975, 2003, incorporated by reference herein in its entirety. Most of the useful modifications to the siRNA molecules can be introduced using chemistries established for antisense oligonucleotide technology. In one embodiment, the modifications involve minimal 2′-O-methyl modification, preferably excluding such modification. Modifications also preferably exclude modifications of the free 5′-hydroxyl groups of the siRNA.

In some embodiments, the compound provided in the compositions and delivered via the methods described herein is a microRNA (miR). The miR can be an endogenous miR or artificial miR (referred to herein as a miRNA mimic or miR-mim). An endogenous miR is a small RNA naturally present in the genome which is capable of modulating the productive utilization of mRNA. An artificial miR includes any type of RNA sequence, other than endogenous miR, which is capable of modulating the productive utilization of mRNA.

In some embodiments, the miR is miR-660 (SEQ ID NO:2 or SEQ ID NO:3) or a functional variant thereof. In some embodiments, the miR functional variant is at least 98% identical in sequence to either SEQ ID NO:2 or SEQ ID NO:3. In some embodiments, the miR functional variant is at least 95% identical in sequence to SEQ ID NO:2 or SEQ ID NO:3. In some embodiments, the miR functional variant is at least 90% identical in sequence to either SEQ ID NO:2 or SEQ ID NO:3. In some embodiments, the miR functional variant is at least 85% identical in sequence to SEQ ID NO:2 or SEQ ID NO:3. In some embodiments, the miR functional variant is at least 80% identical in sequence to SEQ ID NO:2 or SEQ ID NO:3.

In yet another embodiment of the invention, the compound that reduces the expression level of MDM2 is an antisense oligonucleotide complementary to the MDM2 gene.

In some embodiments, the compound comprises the sequence 5′-ACCCAUU-3′ (SEQ ID NO: 4) or 5′-ACCCATT-3′ (SEQ ID NO: 5). As established in the Example herein, a miR-660 (SEQ ID NO:2) targets the sequence 5′-AAUGGGU-3′ (SEQ ID NO: 6) on MDM2, through the miR-660 complementary sequence 5′-ACCCAUU-3′ (SEQ ID NO: 4). That MDM2 sequence is thus an effective antisense target and other antisense molecules that have 5′-ACCCAUU-3′ (SEQ ID NO: 4) or 5′-ACCCATT-3′ (SEQ ID NO: 5) can be effective in reducing expression of MDM2. Thus, the present invention in one embodiment includes a composition comprising an MDM2 antisense inhibitor comprising the sequence 5′-ACCCAUU-3′ (SEQ ID NO: 4) or 5′-ACCCATT-3′ (SEQ ID NO: 5).

In some embodiments, the MDM2 antisense inhibitor comprises one or more modified nucleotides such that the antisense inhibitor is stable in a human longer than an antisense inhibitor that does not comprise the one or more modified nucleotides. In other embodiments, the MDM2 antisense inhibitor comprises SEQ ID NO: 2 or SEQ ID NO: 3, or a functional variant thereof.

In some embodiments, the compositions provided herein comprise a miR-660 comprising one or more modified nucleotides such that the miR-660 is stable in a human longer than a miR-660 that does not comprise the one or more modified nucleotides.

In one embodiment, the nucleic acid compound includes a modification to one or more of the nucleotides. Modifications can be made to the nucleic acid such that the modified nucleic acid is stable in a human longer than the nucleic acid that does not comprise said modification. For example, the nucleic acid compound can be modified to comprise one or more modified nucleotides (e.g., 1, 2, 3, 4, or more). Methods of modifying a nucleic acid to enhance its stability are known in the art, for example, such as those disclosed in U.S. Pat. No. 7,579,451, U.S. Patent Publications 2014/0179771 and 2014/0179763, the contents of each of which are incorporated herein by reference. In some embodiments provided herein, the compound is a miR-660 comprising one or more modified nucleotides such that the miR-660 is stable in a human longer than a miR-660 that does not comprise the one or more modified nucleotides.

In some embodiments, the compound is a peptide or peptidomimetic, or a small molecule which inhibits activity of the MDM2 protein, e.g., a small molecule which inhibits a protein-protein interaction between the MDM2 protein and p53, or an aptamer which inhibits expression or activity of the MDM2 protein.

In some embodiments, the compound described herein is encoded by a vector such as a viral vector or plasmid based expression vector. After the administration of the vector to the patient, the compound in one embodiment is expressed endogenously inside the patient. As used herein, the term “vector” refers to a nucleic acid molecule capable of transporting another nucleic acid to which it has been linked. In one embodiment, the vector includes a polyadenylation sequence, one or more restriction sites, as well as one or more selectable markers such as neomycin phosphotransferase, hygromycin phosphotransferase or puromycin-Nacetyl-transferase. Additionally, depending on the host cell chosen and the vector employed, other genetic elements such as an origin of replication, additional nucleic acid restriction sites, enhancers, sequences conferring inducibility of transcription, and selectable markers, may also be incorporated into the vectors described herein.

Examples of commercially available plasmid-based expression vectors for RNAi compounds include but are not limited to members of the pSilencer series (Ambion, Austin, Tex.) and pCpG-siRNA (InvivoGen, San Diego, Calif.). Viral vectors for expression of interfering RN may be derived from a variety of viruses including adenovirus, adeno-associated virus, lentivirus (e.g., HIV, FIV, and EIAV), and herpes virus. Examples of commercially available viral vectors for shRNA expression include pSilencer adeno (Ambion, Austin, Tex.) and pLenti6/BLOCKiT™-DEST (Invitrogen, Carlsbad, Calif.). Selection of viral vectors, methods for expressing the interfering RNA from the vector and methods of delivering the viral vector are within the ordinary skill of the art. Examples of kits for production of PCR-generated shRNA expression cassettes include Silencer Express (Ambion, Austin, Tex.) and siXpress (Mirus, Madison, Wis.).

One type of vector is a “plasmid”, which refers to a circular double stranded DNA loop into which additional nucleic acid segments can be ligated. Another type of vector is a viral vector, wherein additional nucleic acid segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors”, or more simply “expression vectors.” In general, expression vectors of utility in recombinant DNA techniques are often in the form of plasmids. Non-limiting examples of viral vectors include replication defective retroviruses, lentiviruses, adenoviruses and adeno-associated viruses (AAV). In some embodiments, a vector capable of expressing miR-660 when transfected into a human cell is provided. In some of these embodiments, the vector is a viral vector.

In various embodiments, the miR-660 is administered to the patient such that it is expressed from a vector in a cell of the patient. See, e.g., Example, using a lentiviral vector. Non-limiting examples of other vectors include other engineered viruses, plasmids, and mammalian expression vectors.

Importantly, and as evident from the disclosure set forth above, the compositions of the invention and the methods for treating a cancer patient, e.g., by reducing MDM2 expression or MDM2 activity level or MDM2 interaction with p53 in the patient's tumor tissue (e.g., lung tumor tissue) are not limited to any particular compound. Examples of such treatments include use of small molecule inhibitors (see, e.g., Zhao et al., 2013), peptides (U.S. Pat. No. 8,598,127), antibodies (Weisbart et al., 2012), antisense (see, e.g., Chen et al., 1999, using phosphorothionate nucleotide analogs), or administration of RNAi compounds such as miR-660.

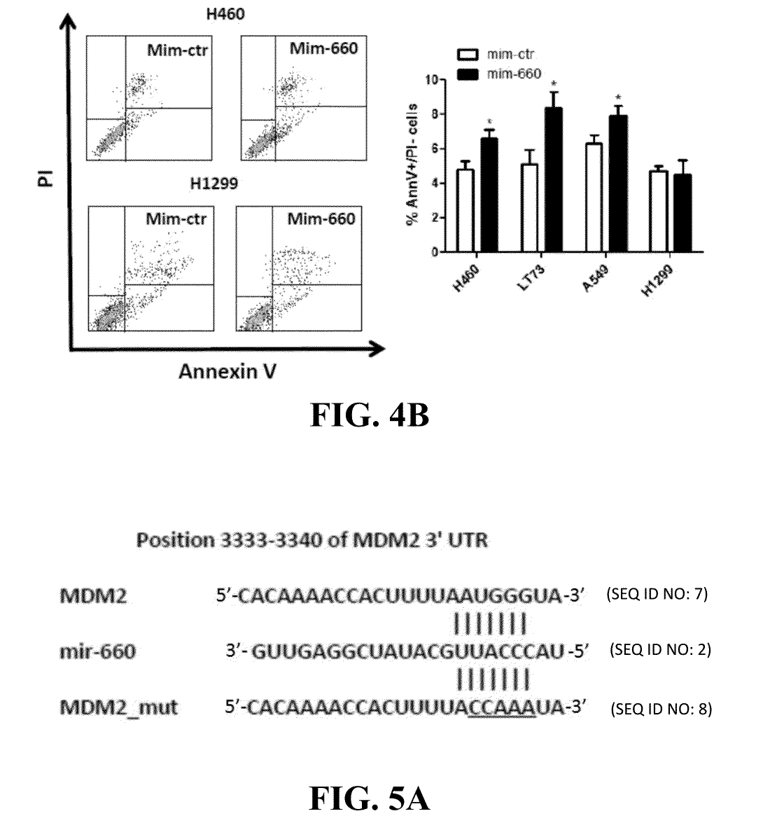

Without wishing to be bound by theory, the administration of a miR-660 (e.g., of SEQ ID NO: 2) can be considered to be an MDM2 antisense treatment. In one embodiment, the miR-660, e.g., of SEQ ID NO: 2 targets a specific sequence (AAUGGGU (SEQ ID NO: 6)) in the 3′UTR of MDM2 mRNA (see Example below and FIGS. 4A-4B). Thus, an antisense molecule having the sequence ACCCAUU (SEQ ID NO: 4) (or ACCCATT (SEQ ID NO: 5) for DNA) without wishing to be bound by theory, would likely inhibit MDM2 production (and MDM2-mediated inhibition of p53) since miR-660 inhibits MDM2 production with that sequence.

As provided throughout, in one aspect, the present invention provides a pharmaceutical composition comprising the compound described herein and a pharmaceutically acceptable carrier. In one embodiment, the pharmaceutical composition comprises miR-660 or a functional variant thereof, e.g., a variant having one or more modified nucleotides, and a pharmaceutically acceptable carrier.

Pharmaceutically acceptable carriers amenable for use herein are covalently or non-covalently bound, admixed, encapsulated, conjugated, operably-linked, or otherwise associated with the therapeutic agent such that the pharmaceutically acceptable carrier stabilizes or increases the cellular uptake, stability, solubility, half-life, binding efficacy, specificity, targeting, distribution, absorption, or renal clearance of the agent. Alternatively, or in addition, the pharmaceutically acceptable carrier increases or decreases the immunogenicity of the compound or allows for greater ease of administration of the compound. In one embodiment, the pharmaceutically acceptable carrier may be capable of increasing the cytotoxicity of the agent with respect to the targeted cancer cells.

Alternatively, or in addition, a pharmaceutically acceptable carrier amenable for use herein is a salt (for example, acid addition salts, e.g., salts of hydrochloric, hydrobromic, acetic acid, and benzene sulfonic acid), esters, salts of such esters, or any other compound which, upon administration to a subject, are capable of providing (directly or indirectly) the biologically active compositions of the invention. As such, the invention encompasses prodrugs, and other bioequivalents. As used herein, the term “prodrug” is a pharmacological substance that is administered in an inactive (or significantly less active) form. Once administered, the prodrug is metabolized in vivo into an active metabolite. Pharmaceutically acceptable carriers are alternatively or additionally diluents, excipients, adjuvants, emulsifiers, buffers, stabilizers, and/or preservatives.

Pharmaceutically acceptable carriers of the invention are therapeutic agent delivery systems/mechanisms that increase uptake of the agent by targeted cells. Non-limiting examples of pharmaceutically acceptable carriers include liposomes, cationic lipids, anionic lipids, cationic polymers, polymers, hydrogels, micro- or nano-capsules (biodegradable), microspheres (optionally bioadhesive), cyclodextrins, or any combination of the preceding elements (see PCT Publication No. WO 00/53722; U.S. Patent Publication 2008/0076701, each of which is incorporated by reference herein in its entirety). Moreover, pharmaceutically acceptable carriers that increase cellular uptake can be modified with cell-specific proteins or other elements such as receptors, ligands, antibodies to specifically target cellular uptake to a chosen cell type.

In one embodiment of the invention, compositions are first introduced into a cell or cell population that is subsequently administered to a subject. In some embodiments, the agent is delivered intracellularly, e.g., in cells of a target tissue such as lung, or in inflamed tissues. Included within the invention are compositions and methods for delivery of the agent composition by removing cells of a subject, delivering the isolated agent composition to the removed cells, and reintroducing the cells into a subject. In some embodiments, the compound described herein (e.g., miR-660) is combined with a cationic lipid or transfection material such as LIPOFECTAMINE (Invitrogen).

In one embodiment, the compound that reduces the activity or expression of MDM2, or the interaction of MDM2 with p53, is prepared with a pharmaceutically acceptable carrier(s) that protects the compound against rapid elimination from the body, such as a controlled release formulation, including implants and microencapsulated delivery systems. Biodegradable, biocompatible polymers can be used as carriers, such as ethylene vinyl acetate, polyanhydrides, polyglycolic acid, collagen, polyorthoesters, and polylactic acid. Methods for preparation of such formulations will be apparent to those skilled in the art. Examples of materials which can form hydrogels include polylactic acid, polyglycolic acid, PLGA polymers, alginates and alginate derivatives, gelatin, collagen, agarose, natural and synthetic polysaccharides, poly-amino acids such as polypeptides particularly poly(lysine), polyesters such as polyhydroxybutyrate and poly-ε-caprolactone, polyanhydrides, polyphosphazines, poly(vinyl alcohols), poly(alkylene oxides) particularly poly(ethylene oxides), poly(allylamines) (PAM), poly(acrylates), modified styrene polymers such as poly(4-aminomethylstyrene), pluronic polyols, polyoxamers, poly(uronic acids), poly(vinylpyrrolidone) and copolymers of the above, including graft copolymers.

Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811, incorporated by reference herein in its entirety.

In one embodiment, a pharmaceutically acceptable carrier is one or more cationic lipids that are bound or associated with the miR-660 or other RNAi compound. Alternatively, or in additionally, the miR-660 (or other RNAi compound) is encapsulated or surrounded in cationic lipids, e.g. liposomes, for in vivo delivery. Exemplary cationic lipids include, but are not limited to, N41-(2,3-dioleoyloxy)propyliN,N,N-trimethylammonium chloride (DOTMA); 1,2-bis(oleoyloxy)-3-3-(trimethylammonium)propane (DOTAP), 1,2-bis(dimyrstoyloxy)-3-3-(trimethylammonia)propane (DMTAP); 1,2-dimyristyloxypropyl-3-dimethylhydroxyethylammonium bromide (DMRIE); dimethyldioctadecylammonium bromide (DDAB); 3-(N—(N′,N′-dimethylaminoethane)carbamoyl)cholesterol (DC-Chol); 3β-[N′,N′-diguanidinoethyl-aminoethane)carbamoyl cholesterol (BGTC); 2-(2-(3-(bis(3-aminopropyl)amino)propylamino)acetamido)-N,N-ditetradecyla-cetamide (RPR209120); pharmaceutically acceptable salts thereof, and mixtures thereof. Further exemplary cationic lipids include, but are not limited to, 1,2-dialkenoyl-sn-glycero-3-ethylphosphocholines (EPCs), such as 1,2-dioleoyl-sn-glycero-3-ethylphosphocholine, 1,2-distearoyl-sn-glycero-3-ethylphosphocholine, 1,2-dipalmitoyl-sn-glycero-3-ethylphosphocholine, pharmaceutically acceptable salts thereof, and mixtures thereof.

Exemplary polycationic lipids include, but are not limited to, tetramethyltetrapalmitoyl spermine (TMTPS), tetramethyltetraoleyl spermine (TMTOS), tetramethlytetralauryl spermine (TMTLS), tetramethyltetramyristyl spermine (TMTMS), tetramethyldioleyl spermine (TMDOS), pharmaceutically acceptable salts thereof, and mixtures thereof. Further exemplary polycationic lipids include, but are not limited to, 2,5-bis(3-aminopropylamino)-N-(2-(dioctadecylamino)-2-oxoethyl)pentanamid-e (DOGS); 2,5-bis(3-aminopropylamino)-N-(2-(di(Z)-octadeca-9-dienylamino)-2-oxoethyl)pentanamide (DOGS-9-en); 2,5-bis(3-aminopropylamino)-N-(2-(di(9Z,12Z)-octadeca-9,12-dienylamino)-2-oxoethyl)pentanamide (DLinGS); 3-beta-(N4-(N1,N8-dicarbobenzoxyspermidine)carbamoyl)cholesterol (GL-67); (9Z,9yZ)-2-(2,5-bis(3-aminopropylamino)pentanamido)propane-1,3-diyl-dioct-adec-9-enoate (DOSPER); 2,3-dioleyloxy-N-[2(sperminecarboxamido)ethyl]-N,N-dimethyl-1-propanamini-urn trifluoro-acetate (DOSPA); pharmaceutically acceptable salts thereof, and mixtures thereof.

Examples of cationic lipids amenable for use with the compositions described herein include but are not limited to those described in U.S. Pat. Nos. 4,897,355; 5,279,833; 6,733,777; 6,376,248; 5,736,392; 5,334,761; 5,459,127; U.S. Patent Application Publication No. 2005/0064595; U.S. Pat. Nos. 5,208,036; 5,264,618; 5,279,833; 5,283,185; 5,753,613; and 5,785,992, the contents of which are incorporated by reference in their entireties for all purposes.

Pharmaceutically acceptable carriers of the invention also include non-cationic lipids, such as neutral, zwitterionic, and anionic lipids. Exemplary non-cationic lipids amenable for use herein include, but are not limited to, 1,2-Dilauroyl-sn-glycerol (DLG); 1,2-Dimyristoyl-snglycerol (DMG); 1,2-Dipalmitoyl-sn-glycerol (DPG); 1,2-Distearoyl-sn-glycerol (DSG); 1,2-Dilauroyl-sn-glycero-3-phosphatidic acid (sodium salt; DLPA); 1,2-Dimyristoyl-snglycero-3-phosphatidic acid (sodium salt; DMPA); 1,2-Dipalmitoyl-sn-glycero-3-phosphatidic acid (sodium salt; DPPA); 1,2-Distearoyl-sn-glycero-3-phosphatidic acid (sodium salt; DSPA); 1,2-Diarachidoyl-sn-glycero-3-phosphocholine (DAPC); 1,2-Dilauroyl-sn-glycero-3-phosphocholine (DLPC); 1,2-Dimyristoyl-sn-glycero-3-phosphocholine (DMPC); 1,2-Dipalmitoyl-sn-glycero-O-ethyl-3-phosphocholine (chloride or triflate; DPePC); 1,2-Dipalmitoyl-sn-glycero-3-phosphocholine (DPPC); 1,2-Distearoyl-sn-glycero-3-phosphocholine (DSPC); 1,2-Dilauroyl-sn-glycero-3-phosphoethanolamine (DLPE); 1,2-Dimyristoyl-sn-glycero-3-phosphoethanolamine (DMPE); 1,2-Dipalmitoyl-sn-glycero-3-phosphoethanolamine (DPPE); 1,2-Distearoylsn-glycero-3-phosphoethanolamine (DSPE); 1,2-Dilauroyl-sn-glycero-3-phosphoglycerol (sodium salt; DLPG); 1,2-Dimyristoyl-sn-glycero-3-phosphoglycerol (sodium salt; DMPG); 1,2-Dimyristoyl-sn-glycero-3-phospho-sn-1-glycerol (ammonium salt; DMP-snl-G); 1,2-Dipalmitoyl-sn-glycero-3-phosphoglycerol (sodium salt; DPPG); 1,2-Distearoyl-sn-glycero-3-phosphoglycero (sodium salt; DSPG); 1,2-Distearoyl-snglycero-3-phospho-sn-1-glycerol (sodium salt; DSP-sn-1-G); 1,2-Dipalmitoyl-snglycero-3-phospho-L-serine (sodium salt; DPP S); 1-Palmitoyl-2-linoleoyl-sn-glycero-3-phosphocholine (PLinoPC); 1-Palmitoyl-2-oleoyl-sn-glycero-3-phosphocholine (POPC); 1-Palmitoyl-2-oleoyl-sn-glycero-3-phosphoglycerol (sodium salt; POPG); 1-Palmitoyl-2-oleoyl-sn-glycero-3-phosphoglycerol (sodium salt; POPG); 1-Palmitoyl-2-oleoyl-snglycero-3-phosphoglycerol (ammonium salt; POPG); 1-Palmitoyl-2-4-o-sn-glycero-3-phosphocholine (P-lyso-PC); 1-Stearoyl-2-lyso-sn-glycero-3-phosphocholine (S-lysoPC); and mixtures thereof. Further exemplary non-cationic lipids include, but are not limited to, polymeric compounds and polymer-lipid conjugates or polymeric lipids, such as pegylated lipids, including polyethyleneglycols, N-(Carbonylmethoxypolyethyleneglycol-2000)-1,2-dimyristoyl-sn-glycero-3-phosphoethanolamine (sodium salt; DMPE-MPEG-2000); N-(Carbonyl-methoxypolyethyleneglycol-5000)-1,2-dimyristoyl-sn-glycero-3-phosphoethanolamine (sodium salt; DMPE-MPEG-5000); N(Carbonyl-methoxypolyethyleneglycol 2000)-1,2-dipalmitoyl-sn-glycero-3-phosphoethanolamine (sodium salt; DPPE-MPEG-2000); N-(Carbonyl-methoxypolyethyleneglycol 5000)-1,2-dipalmitoyl-sn-glycero-3-phosphoethanolamine (sodium salt; DPPE-MPEG-5000); N-(Carbonyl-methoxypolyethyleneglycol 750)-1,2-distearoyl-sn-glycero-3-phosphoethanolamine (sodium salt; DSPE-MPEG-750); N(Carbonyl-methoxypolyethyleneglycol 2000)-1,2-distearoyl-sn-glycero-3-phosphoethanolamine (sodium salt; DSPE-MPEG-2000); N-(Carbonylmethoxypolyethyleneglycol 5000)-1,2-distearoyl-sn-glycero-3-phosphoethanolamine (sodium salt; DSPE-MPEG-5000); sodium cholesteryl sulfate (SCS); pharmaceutically acceptable salts thereof, and mixtures thereof. Examples of non-cationic lipids include, but are not limited to, dioleoylphosphatidylethanolamine (DOPE), diphytanoylphosphatidylethanolamine (DPhPE), 1,2-Dioleoyl-sn-Glycero-3-Phosphocholine (DOPC), 1,2-Diphytanoyl-sn-Glycero-3-Phosphocholine (DPhPC), cholesterol, and mixtures thereof.

Pharmaceutically-acceptable carriers of the invention in one embodiment, include one or more anionic lipids. Exemplary anionic lipids include, but are not limited to, phosphatidylserine, phosphatidic acid, phosphatidylcholine, platelet-activation factor (PAF), phosphatidylethanolamine, phosphatidyl-DL-glycerol, phosphatidylinositol, phosphatidylinositol (pi(4)p, pi(4,5)p2), cardiolipin (sodium salt), lysophosphatides, hydrogenated phospholipids, sphingoplipids, gangliosides, phytosphingosine, sphinganines, pharmaceutically acceptable salts thereof, and mixtures thereof.

Another aspect of the invention relates to the treatment of a cancer patient, e.g., a lung cancer patient, with one or more of the compositions described herein. For example, in one embodiment, a patient in need of treatment of lung cancer is administered a composition comprising a therapeutically effective amount of a composition that reduces the activity or expression of MDM2, or the interaction of MDM2 with p53. In a further embodiment, the compound is an MDM2 RNAi compound. In even a further embodiment, the compound is a miR-660.

Methods for delivering the therapeutic compositions for use herein are described, e.g., in Akhtar, et al., Trends Cell Bio. 2:139, 1992; Delivery Strategies for Antisense Oligonucleotide Therapeutics, ed. Akhtar, 1995; Maurer, et al., Mol. Membr. Biol. 16:129-140, 1999; Hofland and Huang, Handb. Exp. Pharmacol. 137:165-192, 1999; and Lee, et al., ACS Symp. Ser. 752:184-192, 2000, each of which is incorporated by reference herein in its entirety for all purposes. International PCT Publication No. WO 94/02595 further describes general methods for delivery of enzymatic nucleic acid molecules. These protocols can be utilized to supplement or complement delivery of the agent.

Pharmaceutical compositions can be administered locally and/or systemically. As used herein, the term “local administration” is meant to describe the administration of a pharmaceutical composition of the invention to a specific tissue or area of the body with minimal dissemination of the composition to surrounding tissues or areas. Locally administered pharmaceutical compositions are not detectable in the general blood stream when sampled at a site not immediate adjacent or subjacent to the site of administration.

As used herein the term “systemic administration” is meant to describe in vivo systemic absorption or accumulation of drugs in the blood stream followed by distribution throughout the entire body. Administration routes which lead to systemic absorption include, without limitation: intravenous, subcutaneous, intraperitoneal, inhalation, oral, intrapulmonary and intramuscular. Each of these administration routes exposes the therapeutic agent to an accessible diseased tissue. The rate of entry of a drug into the circulation has been shown to be a function of molecular weight or size. The use of a liposome or other drug carrier comprising the compounds of the instant disclosure can potentially localize the drug, e.g., in certain tissue types, such as the tissues of the reticular endothelial system (RES). A liposome formulation that can facilitate the association of drug with the surface of cells, such as, lymphocytes and macrophages is also useful. This approach may provide enhanced delivery of the drug to target cells by taking advantage of the specificity of macrophage and lymphocyte immune recognition of abnormal cells, such as cancer cells.

A pharmaceutically effective dose depends on the type of disease, the composition used, the route of administration, the individual and physical characteristics of the subject under consideration (for example, age, gender, weight, diet, smoking-habit, exercise-routine, genetic background, medical history, hydration, blood chemistry), concurrent medication, and other factors that those skilled in the medical arts will recognize.

Generally, an amount from about 0.01 mg/kg and 25 mg/kg body weight/day of active ingredients is administered dependent upon potency of the miRNA and/or the miRNA inhibitor, e.g. the therapeutic composition. In alternative embodiments dosage ranges include, but are not limited to, 0.01-0.1 mg/kg, 0.01-1 mg/kg, 0.01-10 mg/kg, 0.01-20 mg/kg, 0.01-30 mg/kg, 0.01-40 mg/kg, 0.01-50 mg/kg, 0.01-60 mg/kg, 0.01-70 mg/kg, 0.01-80 mg/kg, 0.01-90 mg/kg, 0.01-100 mg/kg, 0.01-150 mg/kg, 0.01-200 mg/kg, 0.01-250 mg/kg, 0.01-300 mg/kg, 0.01-500 mg/kg, and all ranges and points in between. In alternative embodiments dosage ranges include, but are not limited to, 0.01-1 mg/kg, 1-10 mg/kg, 10-20 mg/kg, 20-30 mg/kg, 30-40 mg/kg, 40-50 mg/kg, 50-60 mg/kg, 60-70 mg/kg, 70-80 mg/kg, 80-90 mg/kg, 90-100 mg/kg, 100-150 mg/kg, 150-200 mg/kg, 200-300 mg/kg, 300-500 mg/kg, and all ranges and points in between.

A pharmaceutically acceptable carrier is chosen to be compatible with its intended route of administration. Examples of routes of administration include parenteral, e.g., intravenous, intradermal, subcutaneous, oral (e.g., inhalation or insufflation), transdermal (topical), transmucosal, transopthalmic, tracheal, intranasal, epidermal, intraperitoneal, intraorbital, intraarterial, intracapsular, intraspinal, intrasternal, intracranial, intrathecal, intraventricular, and rectal administration. Alternatively, or in addition, compositions of the invention are administered non-parentally, for example, orally. Alternatively, or further in addition, compositions of the invention are administered surgically, for example, as implants or biocompatible polymers.

Pharmaceutical compositions are administered via injection or infusion, e.g. by use of an infusion pump. Direct injection of the nucleic acid molecules of the invention, is performed using standard needle and syringe methodologies, or by needle-free technologies such as those described in Conry et al., Clin. Cancer Res. 5:2330-2337, 1999 and International PCT Publication No. WO 99/31262, each of which is incorporated by reference herein in its entirety.

Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfate; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

An isolated agent with a pharmaceutically acceptable carrier of the invention can be administered to a subject in many of the well-known methods currently used for chemotherapeutic treatment. For example, for treatment of cancers, a compound of the invention may be injected directly into tumors, injected into the blood stream or body cavities or taken orally or applied through the skin with patches. The dose chosen should be sufficient to constitute effective treatment but not so high as to cause unacceptable side effects. The state of the disease condition (e.g., cancer, precancer, and the like) and the health of the subject should preferably be closely monitored during and for a reasonable period after treatment.

In one embodiment, compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof.

The pharmaceutical compositions can be in the form of a sterile injectable aqueous or oleaginous suspension. This suspension is formulated according to the known art using those suitable dispersing or wetting agents and suspending agents that have been mentioned above. The sterile injectable preparation is a sterile injectable solution or suspension in a non-toxic parentally acceptable diluent or solvent, e.g., as a solution in 1,3-butanediol. Exemplary acceptable vehicles and solvents are water, Ringer's solution and isotonic sodium chloride solution. In addition, sterile, fixed oils are conventionally employed as a solvent or suspending medium. For this purpose, any bland fixed oil is employed including synthetic mono- or diglycerides. In addition, fatty acids such as oleic acid are used in the preparation of injectables.

Sterile injectable solutions can be prepared by incorporating the agent in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible pharmaceutically acceptable carrier. Agents containing at least one 2′-O-methoxyethyl modification are used when formulating compositions for oral administration. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser, which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Exemplary penetrants for transdermal administration include, but are not limited to, lipids, liposomes, fatty acids, fatty acid, esters, steroids, chelating agents, and surfactants. Preferred lipids and liposomes of the invention are neutral, negative, or cationic. Compositions are encapsulated within liposomes or form complexes thereto, such as cationic liposomes.

Alternatively, or additionally, the compound is complexed to one or more lipids, such as one or more cationic lipids, e.g., in the form of a lipid nanoparticle or liposome. Compositions prepared for transdermal administration in one embodiment are provided by iontophoresis. Such penetrants are generally known in the art, and include, for example, for transmucosal administration, detergents, bile salts, and fusidic acid derivatives.

Transmucosal administration can be accomplished through the use of nasal sprays or suppositories. For transdermal administration, the active compounds are formulated into patches, ointments, lotions, salves, gels, drops, sprays, liquids, powders, or creams as generally known in the art.

Other non-limiting examples of delivery strategies for the therapeutic agents of the instant disclosure include material described in Boado, et al., J. Pharm. Sci. 87:1308-1315, 1998; Tyler, et al., FEBS Lett. 421:280-284, 1999; Pardridge, et al, PNAS USA. 92:5592-5596, 1995; Boado, Adv. Drug Delivery Rev. 15:73-107, 1995; Aldrian-Herrada, et al., Nucleic Acids Res. 26:4910-4916, 1998; and Tyler, et al., PNAS USA. 96:7053-7058, 1999, the contents of each of which are incorporated by reference herein in their entireties.

The compositions of the invention may also be administered in the form of suppositories, e.g., for rectal administration of the drug. These compositions are prepared by mixing the drug with a suitable non-irritating excipient that is solid at ordinary temperatures but liquid at the rectal temperature and will therefore melt in the rectum to release the drug. Such materials include cocoa butter and polyethylene glycols.

Aqueous suspensions contain the active materials in admixture with excipients suitable for the manufacture of aqueous suspensions. Such excipients are suspending agents, e.g., sodium carboxymethylcellulose, methylcellulose, hydropropylmethylcellulose, sodium alginate, polyvinylpyrrolidone, gum tragacanth and gum acacia; dispersing or wetting agents can be a naturally-occurring phosphatide, e.g., lecithin, or condensation products of an alkylene oxide with fatty acids, e.g., polyoxyethylene stearate, or condensation products of ethylene oxide with long chain aliphatic alcohols, e.g., heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, e.g., polyethylene sorbitan monooleate. The aqueous suspensions also contain one or more preservatives, e.g., ethyl, or n-propyl p-hydroxybenzoate, one or more coloring agents, one or more flavoring agents, and one or more sweetening agents, such as sucrose or saccharin.

Oily suspensions are formulated by suspending the active ingredients in a vegetable oil, e.g., arachis oil, olive oil, sesame oil or coconut oil, or in a mineral oil such as liquid paraffin. The oily suspensions contain a thickening agent, e.g., beeswax, hard paraffin or cetyl alcohol. Sweetening agents and flavoring agents are added to provide palatable oral preparations. These compositions are preserved by the addition of an antioxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents or suspending agents are exemplified by those already mentioned above. Additional excipients, e.g., sweetening, flavoring and coloring agents, are also present.

Pharmaceutical compositions of the invention can be in the form of oil-in-water emulsions. The oily phase is a vegetable oil or a mineral oil or mixtures of these. Suitable emulsifying agents are naturally-occurring gums, e.g., gum acacia or gum tragacanth, naturally-occurring phosphatides, e.g., soy bean, lecithin, and esters or partial esters derived from fatty acids and hexitol, anhydrides, e.g., sorbitan monooleate, and condensation products of the said partial esters with ethylene oxide, e.g., polyoxyethylene sorbitan monooleate. The emulsions also contain sweetening and flavoring agents.

In a preferred aspect, the pharmaceutically acceptable carrier can be a solubilizing carrier molecule. More preferably, the solubilizing carrier molecule can be Poloxamer, Povidone K17, Povidone K12, Tween 80, ethanol, Cremophor/ethanol, Lipiodol, polyethylene glycol (PEG) 400, propylene glycol, Trappsol, alpha-cyclodextrin or analogs thereof, beta-cyclodextrin or analogs thereof, and gamma-cyclodextrin or analogs thereof.

The invention also provides compositions prepared for storage or administration. Acceptable carriers or diluents for therapeutic use are well known in the pharmaceutical art, and are described, e.g., in Remington's Pharmaceutical Sciences, Mack Publishing Co., A. R. Gennaro Ed., 1985. For example, preservatives, stabilizers, dyes and flavoring agents are provided. These include sodium benzoate, sorbic acid and esters of phydroxybenzoic acid. In addition, antioxidants and suspending agents are used.

In one aspect, a composition provided herein is administered to a lung cancer patient in need of treatment. The lung cancer, in one embodiment, is small cell lung cancer. In another embodiment, the lung cancer is non-small cell lung cancer (NSCLC). In a further embodiment, the NSCLC is adenocarcinoma, squamous cell carcinoma or large-cell lung cancer.

In one embodiment, the patient in need of treatment (i) expresses miR-660 in a lung tissue sample or biological fluid sample at a level lower than a control level derived from a subject or plurality of subjects that do not have lung cancer, or as compared to a control level derived from a lung cancer patient or plurality thereof, that have been given a favorable prognosis; (ii) expresses MDM2 at a higher level in a lung tissue sample or biological fluid sample as compared to a control level derived from a subject or plurality of subjects that do not have lung cancer, or as compared to a control level derived from a lung cancer patient or plurality thereof that have been given a favorable prognosis; and/or (iii) expresses p53 in a lung tissue sample or a biological fluid sample below a control level derived from a lung tumor tissue sample (or plurality thereof) or a biological fluid sample (or plurality thereof) obtained from a patient that has a favorable lung cancer prognosis; or a control level derived from a healthy subject.

In one embodiment of the methods of treatments described herein, a patient in need of treatment expresses p53 in a lung cancer tumor sample obtained from the patient. In a further embodiment, the patient is in need of treatment if the MDM2 expression level in the patient's sample of lung tumor tissue is higher than an MDM2 expression level in a non-cancerous tissue sample obtained from the patient, or obtained from a different patient. In one embodiment, a patient in need of treatment expresses p53 in his or her lung tumor tissue sample below a p53 expression level in a tissue sample from non-cancerous tissue obtained from the patient (a control level).

In another embodiment, a patient is in need of treatment and therefore administered one of the compositions provided herein if the patient's lung tumor tissue has one or more of the following characteristics (i) p53 is present in a lung tumor tissue sample from the patient; (ii) the MDM2 expression level in the tumor tissue sample from the patient is above an MDM2 expression level in non-cancerous tissue; (iii) the miR-660 concentration in the patient in a lung tissue sample or biological fluid sample is below a miR-660 mean concentration control level derived from lung tissue samples or biological fluid samples from a plurality of control subjects that do not have lung cancer, or a plurality of lung cancer patients that have been given a favorable prognosis.

In some embodiments, the patient in need thereof is selected for treatment based on the MDM2 expression level in a lung tumor tissue sample obtained from the patient or a biological fluid sample obtained from the patient. The biological fluid sample, in one embodiment, is blood, serum, plasma, urine, saliva, mucus, tears, amniotic fluid, breast milk, sputum, cerebrospinal fluid, peritoneal fluid, pleural fluid, seminal fluid, a fraction thereof or a combination thereof. In some embodiments, the MDM2 expression level in a lung tumor tissue sample of the patient is above an MDM2 expression level in a lung tumor tissue sample from a plurality of lung cancer patients that have a favorable prognosis. In some embodiments, the MDM2 expression level in a lung tumor tissue sample of the patient is above an MDM2 expression level in a noncancerous lung tissue sample from the patient. In one embodiment, the MDM2 expression level in a lung tumor tissue sample of the patient is above an MDM2 expression level in a lung tissue sample from a healthy subject. In some embodiments, the MDM2 expression level in a lung tumor tissue sample of the patient is above an average MDM2 expression level in lung tissue samples from a plurality of healthy subjects.

In some embodiments, the patient in need thereof is selected for treatment based on the miR-660 expression level in a lung tumor tissue sample or a biological fluid sample obtained from the patient. The biological fluid sample, in one embodiment, is blood, serum, plasma, urine, saliva, mucus, tears, amniotic fluid, breast milk, sputum, cerebrospinal fluid, peritoneal fluid, pleural fluid, seminal fluid, a fraction thereof or a combination thereof.

In some embodiments, the miR-660 expression level in a lung tumor tissue sample of the patient is below a miR-660 expression level in a noncancerous lung tissue sample from the patient. In some embodiments, the miR-660 expression level in a lung tumor tissue sample of the patient is below a miR-660 expression level in a lung tissue sample of a subject that does not have lung cancer (e.g., a healthy subject). In some embodiments, the miR-660 expression level in a lung tumor tissue sample of the patient is below an average miR-660 expression level in lung tissue samples of a plurality of subjects that do not have lung cancer.

In some embodiments of this method, the miR-660 concentration is also determined in the lung tumor tissue, and, if the miR-660 concentration in the patient is below a miR-660 concentration in the same fluid or tissue of a plurality of lung cancer patients that had a favorable prognosis, the treatment of the patient is recommended, or the patient is treated by administering miR-660 to the patient in a manner sufficient to treat the lung cancer.

In some embodiments, the patient in need thereof id selected based on the miR-660 expression level in a plasma or blood sample obtained from the patient. In some embodiments, the miR-660 expression level in a plasma or blood sample of the patient is below a miR-660 expression level in a plasma or blood sample of a subject that does not have lung cancer (e.g., a healthy subject). In some embodiments, the miR-660 expression level in a plasma sample of the patient is below an average miR-660 expression level in plasma samples of a plurality of subjects that do not have lung cancer.

Accordingly, in some embodiments, the methods for treating cancer provided herein, e.g., lung cancer, comprise treating the patient in such a manner as to increase the miR-660 concentration in the patient's lung tumor tissue, e.g., by administering a miR-660 to the patient in need thereof. In some embodiments, the above treatment methods further comprise recommending treatment, or treating the patient with a therapy that increases miR-660 concentration in the patient's lung tumor tissue, if the patient has lung cancer. Non-limiting examples of such a therapy includes administration of miR-660 and administration of a transient or stable vector that expresses miR-660.

Since miR-660 administration (and other therapies that reduce MDM2 expression and/or activity, and/or MDM2 interaction with p53) is effective against lung tumors when p53 is present (see Example), a determination that p53 is present in the tumor tissue in one embodiment indicates that the patient could benefit from one of the compositions described herein, for example a composition comprising a therapeutically effective amount of miR-660, to raise p53 to normal levels or above. In one embodiment, p53 expression in a patient in need of treatment is a p53 expression level below normal p53 levels or below levels in patients that have a favorable prognosis.

Accordingly, in some embodiments, the patient in need of treatment is identified or selected based on the p53 expression level in a lung tumor tissue sample obtained from the patient. In some embodiments, the lung tumor tissue sample from the patient expresses p53. In some embodiments, the p53 expression level in a lung tumor tissue sample of the patient is below a p53 expression level in a noncancerous lung tissue sample from the patient. In some embodiments, the p53 expression level in a lung tumor tissue sample of the patient is below a p53 expression level in a lung tissue sample of a subject that does not have lung cancer (e.g., a healthy subject). In some embodiments, the p53 expression level in a lung tumor tissue sample of the patient is below an average p53 expression level in lung tissue samples of a plurality of subjects that do not have lung cancer or a plurality of lung cancer patients that have been given a favorable prognosis.

In some embodiments, the patient in need thereof can be selected based on the p53 expression level in a biological fluid sample obtained from the patient. The biological fluid sample, in one embodiment, is blood, serum, plasma, urine, saliva, mucus, tears, amniotic fluid, breast milk, sputum, cerebrospinal fluid, peritoneal fluid, pleural fluid, seminal fluid, a fraction thereof or a combination thereof. In some embodiments, the biological fluid sample from the patient expresses p53. In some embodiments, the p53 expression level in a biological fluid sample of the patient is below a p53 expression level in a plasma sample of a subject that does not have lung cancer (e.g., a healthy subject). In some embodiments, the p53 expression level in a biological fluid sample of the patient is below an average p53 expression level in plasma samples of a plurality of subjects that do not have lung cancer.

In some embodiments, the patient in need of treatment is selected based on his or her p53 expression level and his or her miR-660 expression level. In some embodiments, the patient in need thereof is selected based on the MDM2 expression level and the miR-660 expression level. In some embodiments, the patient in need thereof can be selected based on the p53 expression level and the MDM2 expression level. In some embodiments, the patient in need of treatment is selected based on the expression levels of p53, MDM2, and miR-660.

In some embodiments of this method, the patient is treated with the therapy if the concentration is below a miR-660 concentration in the same fluid or tissue of a plurality of lung cancer patients that had a favorable prognosis. In other embodiments, the therapy that reduces the MDM2 expression level is administration of miR-660. In further embodiments, the patient is treated if the p53 expression level is below a p53 expression level in a tissue sample from non-cancerous tissue, either from the patient or from another subject.

Patients can be selected for treatment with the compositions and methods described herein, for example, via one of the diagnostic methods provided in U.S. Patent Application Publication No. 2015/0191794, the contents of which are incorporated by reference in their entirety for all purposes.

Specifically, U.S. Patent Application Publication No. 2015/0191794 discloses 24 miRNAs and subsets thereof that can be assessed to determine the presence of a pulmonary tumor in a subject. These 24 miRNAs are shown in Table A. In one embodiment, expression of five or more, ten or more, 15 or more, 20 or more or the 24 miRNAs shown in Table A are used to select a subject for treatment with one of the compositions described herein.

TABLE A

| | | |
|---|---|---|
| hsa-miR-16 | hsa-miR-320 | hsa-miR-148a |
| hsa -miR-17 | hsa -miR-451 | hsa -miR-15b |
| hsa-miR-21 | hsa-miR-660 | hsa-miR-19b |
| hsa -miR-101 | hsa -miR-106a | hsa -miR-28-3p |
| hsa-miR-126 | hsa-miR-133a | hsa-miR-30b |
| hsa -miR-145 | hsa -miR-140-3p | hsa -miR-30c |
| hsa-miR-197 | hsa-miR-140-5p | hsa-miR-486-5p |
| hsa -miR-221 | hsa -miR-142-3p | hsa -miR-92a |

A summary of the miRNAs that can be used can be found in Table 2 of the U.S. Patent Application Publication No. 2015/0191794.

For example, in one embodiment, a reverse transcription polymerase chain reaction (RT-PCR) is performed on the at least 5 miRNAs with DNA primers and or DNA probes specific for the at least 5 miRNAs to form cDNA molecules that correspond to the at least 5 miRNAs. The levels of the cDNA molecules that correspond to the at least 5 miRNAs are then detected. Next, the levels of the cDNA molecules that correspond to the at least 5 miRNAs are compared to a sample training set. The sample training set in one embodiment comprises levels of the cDNA molecules that correspond to the at least 5 miRNAs from a reference normal sample or a reference lung cancer sample (e.g., a reference NSCLC sample). The patient is selected for treatment based on the results of this comparison.

In another embodiment, the patient in need of treatment is identified by determining the expression ratio of a miRNA pair (or a plurality thereof) in a biological sample from the patient. The expression ratio is compared with a cut-off value determined from the average ratio of a plurality of corresponding miRNA pairs from a plurality of control samples. Based on this comparison, a positive score for the expression ratio of the miRNA pair is assigned if the ratio exceeds the cut-off value. Additional miRNA pairs are analyzed in an identical fashion, in one embodiment, until either (1) at least nine miRNA pair expression ratios are assigned a positive score, or (2) after the comparison of the expression ratios of 27 miRNA pairs, less than nine miRNA pair expression ratios are assigned a positive score. In one embodiment, based on the number of positive scores, i.e., greater than or equal to nine, the patient is selected for therapy. The miRNA pairs, in one embodiment, include 106a/140-5p, 106a/142-3p, 126/140-5p, 126/142-3p, 133a/142-3p, 140-5p/17, 142-3p/148a, 142-3p/15b, 142-3p/17, 142-3p/21, 142-3p/221, 142-3p/30b, and/or 320/660, or the inverse ratios thereof. A lung cancer tumor is identified (and a patient is selected for treatment) if at least nine of the miRNA pair expression ratios are assigned a positive score. In another embodiment, the patient is not selected for therapy if less than nine miRNA pair expression ratios are assigned a positive score.

In some embodiments, the miRNA pairs include 106a/660, 106a/92a, 126/660, 140-5p/197, 140-5p/28-3p, 142-3p/145, 142-3p/197, 142-3p/28-3p, 17/660, 17/92a, 197/660, 197/92a, 19b/660, 28-3p/660, or the inverse ratios thereof.

For the evaluation of diagnostic and prognostic tests, predictive values help interpret the results of tests in the clinical setting. The diagnostic or prognostic value of a procedure is defined by its sensitivity, specificity, predictive value and efficiency. Any test method will produce True Positive (TP), False Negative (FN), False Positive (FP), and True Negative (TN). The “sensitivity” of a test is the percentage of all patients with disease present or that do respond who have a positive test or (TP/TP+FN)×100%. The “specificity” of a test is the percentage of all patients without disease or who do not respond, who have a negative test or (TN/FP+TN)×100%. The “predictive value” or “PV” of a test is a measure (%) of the times that the value (positive or negative) is the true value, i.e., the percent of all positive tests that are true positives is the Positive Predictive Value (PV+) or (TP/TP+FP)×100%. The “negative predictive value” (PV) is the percentage of patients with a negative test who will not respond or (TN/FN+TN)×100%. The “accuracy” or “efficiency” of a test is the percentage of the times that the test give the correct answer compared to the total number of tests or (TP+TN/TP+TN+FP+FN)×100%. The “error rate” calculates from those patients predicted to respond who did not and those patients who responded that were not predicted to respond or (FP+FN/TP+TN+FP+FN)×100%. The overall test “specificity” is a measure of the accuracy of the sensitivity and specificity of a test do not change as the overall likelihood of disease changes in a population, the predictive value does change. The PV changes with a physician's clinical assessment of the presence or absence of disease or presence or absence of clinical response in a given patient.

A predetermined “cutoff value” can be determined without undue experimentation for any particular parameter, by evaluating patient sampling, treatment conditions and desired sensitivity and specificity. When numerical values from an individual test subject falls below the cutoff value, the subject is classified as falling into a one group or category, e.g. healthy, and when the numerical value, score, or ratio falls above the cutoff, the subject is classified in the alternative group or category.

For any given test, the TP, FN, FP and TN can be determined and adjusted by the skilled artisan without undue experimentation by, e.g., adjusting the cutoff value, adjusting the statistical significance (e.g., the P value) for making a prognostic or diagnostic determination; adjusting the accuracy of the test procedures, etc.

As demonstrated in the Example below, the concentration of miR-660 is decreased in tissues or bodily fluids of lung cancer patients that have a poor prognosis, when compared to the concentration in patients that have a favorable prognosis (FIG. 2A). Without being bound to any particular mechanism, it is believed that the patient has a poor prognosis as a consequence of the decreased miR-660, since miR-660 interacts with MDM2, reducing MDM2 ability to down-regulate p53, and allowing p53 to suppress the tumor.

Thus, in some embodiments, a method of evaluating the prognosis of a lung cancer patient is provided. The method comprises measuring the concentration of miR-660 in a tissue sample or bodily fluid of the patient, wherein a miR-660 concentration in the patient below a miR-660 concentration in the same fluid or tissue of a plurality of lung cancer patients that had a favorable prognosis indicates that the patient has a poor prognosis. In some of these methods, as described below, expression levels of MDM2 and/or p53 are measured as well.

In some embodiments of this method, the miR-660 expression level is measured in a tissue sample of the patient. MDM2 and/or p53 expression levels can also be determined from a tissue sample. With most of these methods, the tissue sample is a tumor tissue sample.

In some embodiments of this method, the miR-660 expression level is measured in a biological fluid sample obtained from the patient. Examples of biological fluid samples include, but are not limited to, blood, serum, plasma, urine, saliva, mucus, tears, amniotic fluid, breast milk, sputum, cerebrospinal fluid, peritoneal fluid, pleural fluid, seminal fluid, a fraction thereof, or a combination thereof.

As used herein, a “tumor sample”, “tumor tissue sample”, “tumor containing sample”, “tumor cell containing sample”, or variations thereof, refer to cell containing samples of tissue or fluid isolated from an individual suspected of being afflicted with, or at risk of developing, cancer. The samples may contain tumor cells which may be isolated by known methods or other appropriate methods as deemed desirable by the skilled practitioner. These include, but are not limited to, microdissection, laser capture microdissection (LCM), or laser microdissection (LMD) before use in the instant disclosure. Alternatively, undissected cells within a “section” of tissue may be used. Non-limiting examples of such samples include primary isolates (in contrast to cultured cells) and may be collected by any non-invasive, minimally invasive, or invasive means, including, but not limited to, ductal lavage, fine needle aspiration, needle biopsy, bronchoscopy, core biopsy, thoracentesis, thoracotomy, the devices and methods described in U.S. Pat. No. 6,328,709, or any other suitable means recognized in the art.

For practicing the invention methods, the tissue can be prepared in any manner that maintains the miRNA or protein concentration at least until the sample is utilized in the invention methods. Non-limiting useful tissue preparations include fresh, fresh-frozen, and formalin fixed/paraffin embedded (“FFPE”). See U.S. Pat. Nos. 7,364,846, 7,723,039, 7,879,555 and 8,012,688 for methods that are useful in evaluating FFPE samples, each of these patents is incorporated by reference in its entirety.

In additional embodiments, the invention methods may be practiced by analyzing miRNA and/or mRNA from single cells or homogenous cell populations which have been dissected away from, or otherwise isolated or purified from, contaminating cells of a sample as present in a simple biopsy. One advantage provided by these embodiments is that contaminating, non-tumor cells (such as infiltrating lymphocytes or other immune system cells) may be removed as so be absent from affecting the nucleic acids identified or the subsequent analysis of miRNA or mRNA expression levels as provided herein. Such contamination is present where a biopsy is used to generate gene expression profiles.

In various embodiments of the invention methods, miR-660 or mRNA is measured in a bodily fluid of a patient. These embodiments are not narrowly limited to any particular bodily fluid, since miRNA and mRNA is present in essentially all bodily fluids (Weber et al., 2010; De Guire et al., 2013; see also Rodriguez-Dorantes et al. 2014). Examples of useful bodily fluids are peripheral blood, serum, plasma, ascites, urine, sputum, saliva, broncheoalveolar lavage fluid, cyst fluid, pleural fluid, peritoneal fluid, lymph, pus, lavage fluids from sinus cavities, bronchopulmonary aspirates, and bone marrow aspirates. The skilled artisan can determine, without undue experimentation, whether any particular bodily fluid is useful for any particular application.

The concentration of the miRNAs provided herein (e.g., of SEQ ID NOs: 2-3, Table A, miR-660, MDM2, p53, etc.) can be determined by any reliable, sensitive, and specific method known in the art. In some embodiments, the protein level of miR-660, or MDM2 or p53 is measured. In some embodiments, the mRNA level miR-660, or MDM2 or p53 is measured.

In some embodiments of the invention methods, miR-660, or MDM2 or p53 mRNA, is amplified prior to measurement. In other embodiments, the level of those nucleic acids is measured during the amplification process. In still other methods, the nucleic acids are not amplified prior to measurement.

Many methods exist for amplifying miRNA nucleic acid sequences such as mature miRNAs, precursor miRNAs, and primary miRNAs, as well as mRNAs. Suitable nucleic acid polymerization and amplification techniques include reverse transcription (RT), polymerase chain reaction (PCR), real-time PCR (quantitative PCR (q-PCR)), nucleic acid sequence-base amplification (NASBA), ligase chain reaction, multiplex ligatable probe amplification, invader technology (Third Wave), rolling circle amplification, in vitro transcription (IVT), strand displacement amplification, transcription-mediated amplification (TMA), RNA (Eberwine) amplification, and other methods that are known to persons skilled in the art. In certain embodiments, more than one amplification method is used, such as reverse transcription followed by real time quantitative PCR (qRT-PCR).

In PCR and q-PCR methods, for example, a set of primers is used for each target sequence. In certain embodiments, the lengths of the primers depends on many factors, including, but not limited to, the desired hybridization temperature between the primers, the target nucleic acid sequence, and the complexity of the different target nucleic acid sequences to be amplified. In certain embodiments, a primer is about 15 to about 35 nucleotides in length. In other embodiments, a primer is equal to or fewer than 15, 20, 25, 30, or 35 nucleotides in length. In additional embodiments, a primer is at least 35 nucleotides in length.

In a further aspect, a forward primer can comprise at least one sequence that anneals to a miRNA or mRNA and alternatively can comprise an additional 5′ non-complementary region. In another aspect, a reverse primer can be designed to anneal to the complement of a reverse transcribed miRNA or mRNA. The reverse primer may be independent of the miRNA or mRNA sequence, and multiple miRNA and mRNA biomarkers may be amplified using the same reverse primer. Alternatively, a reverse primer may be specific for a miRNA or mRNA biomarker.

The qRT-PCR reaction may further be combined with the reverse transcription reaction by including both a reverse transcriptase and a DNA-based thermostable DNA polymerase. When two polymerases are used, a “hot start” approach may be used to maximize assay performance (U.S. Pat. Nos. 5,411,876 and 5,985,619, incorporated by reference in their entireties). For example, the components for a reverse transcriptase reaction and a PCR reaction may be sequestered using one or more thermoactivation methods or chemical alteration to improve polymerization efficiency (U.S. Pat. Nos. 5,550,044, 5,413,924, and 6,403,341, incorporated by reference in their entireties).

In certain embodiments, labels, dyes, or labeled probes and/or primers are used to detect amplified or unamplified miRNAs or mRNAs. The skilled artisan will recognize which detection methods are appropriate based on the sensitivity of the detection method and the abundance of the target. Depending on the sensitivity of the detection method and the abundance of the target, amplification may or may not be required prior to detection. One skilled in the art will recognize the detection methods where miRNA or mRNA amplification is employed.

A probe or primer may include Watson-Crick bases or modified bases. Modified bases include, but are not limited to, the AEGIS bases (from Eragen Biosciences), which have been described, e.g., in U.S. Pat. Nos. 5,432,272, 5,965,364, and 6,001,983, incorporated by reference in their entireties. In certain aspects, bases are joined by a natural phosphodiester bond or a different chemical linkage. Different chemical linkages include, but are not limited to, a peptide bond or a Locked Nucleic Acid (LNA) linkage, which is described, e.g., in U.S. Pat. No. 7,060,809, incorporated by reference in its entirety.

In a further embodiment, oligonucleotide probes or primers present in an amplification reaction are suitable for monitoring the amount of amplification product produced as a function of time. In certain aspects, probes having different single stranded versus double stranded character are used to detect the nucleic acid. Probes include, but are not limited to, the 5′-exonuclease assay (e.g., TaqMan™) probes (see U.S. Pat. No. 5,538,848, incorporated by reference in its entirety), stem-loop molecular beacons (see, e.g., U.S. Pat. Nos. 6,103,476 and 5,925,517, incorporated by reference in their entireties), stemless or linear beacons (see, e.g., WO 9921881, U.S. Pat. Nos. 6,485,901 and 6,649,349), peptide nucleic acid (PNA) Molecular Beacons (see, e.g., U.S. Pat. No. 6,355,421, incorporated by reference in its entirety and U.S. Pat. No. 6,593,091, incorporated by reference in its entirety), linear PNA beacons (see, e.g. U.S. Pat. No. 6,329,144, incorporated by reference in its entirety), non-FRET probes (see, e.g., U.S. Pat. No. 6,150,097, incorporated by reference in its entirety), Sunrise™/AmplifluorB™ probes (see, e.g., U.S. Pat. No. 6,548,250, incorporated by reference in its entirety), stem-loop and duplex Scorpion™ probes (see, e.g., U.S. Pat. No. 6,589,743, incorporated by reference in its entirety), bulge loop probes (see, e.g., U.S. Pat. No. 6,590,091, incorporated by reference in its entirety), pseudo knot probes (see, e.g., U.S. Pat. No. 6,548,250, incorporated by reference in its entirety), cyclicons (see, e.g., U.S. Pat. No. 6,383,752, incorporated by reference in its entirety), MGB Eclipse™ probe (Epoch Biosciences), hairpin probes (see, e.g., U.S. Pat. No. 6,596,490, incorporated by reference in its entirety), PNA light-up probes, antiprimer quench probes (Li et al., Clin. Chem. 53:624-633 (2006), incorporated by reference in its entirety), self-assembled nanoparticle probes, and ferrocene-modified probes described, for example, in U.S. Pat. No. 6,485,901, incorporated by reference in its entirety.

In certain embodiments, one or more of the primers in an amplification reaction can include a label. In yet further embodiments, different probes or primers comprise detectable labels that are distinguishable from one another. In some embodiments a nucleic acid, such as the probe or primer, may be labeled with two or more distinguishable labels.

In some embodiments, the concentration of miR-660 and/or MDM2 and/or p53 mRNA and/or one or more other miRNAs provided herein, e.g., one or more miRNAs from the panel set forth at Table A, is measured using a microarray or another support.

A “microarray” is a linear or two-dimensional or three dimensional (and solid phase) array of discrete regions, each having a defined area, formed on the surface of a solid support such as, but not limited to, glass, plastic, or synthetic membrane. The density of the discrete regions on a microarray is determined by the total numbers of immobilized polynucleotides to be detected on the surface of a single solid phase support, such as of at least about 50/cm², at least about 100/cm², or at least about 500/cm², up to about 1,000/cm² or higher. The arrays may contain less than about 500, about 1000, about 1500, about 2000, about 2500, or about 3000 immobilized polynucleotides in total. As used herein, a DNA microarray is an array of oligonucleotide or polynucleotide probes placed on a chip or other surfaces used to hybridize to amplified or cloned polynucleotides from a sample. Since the position of each particular group of probes in the array is known, the identities of a sample polynucleotides can be determined based on their binding to a particular position in the microarray.

As an alternative to the use of a microarray, an array of any size on a support may be used in the practice of the disclosure, including an arrangement of one or more position of a two-dimensional or three-dimensional arrangement to detect expression of miR-660 or an mRNA.

In some aspects, a label is attached to one or more probes and has one or more of the following properties: (i) provides a detectable signal; (ii) interacts with a second label to modify the detectable signal provided by the second label, e.g., FRET (Fluorescent Resonance Energy Transfer); (iii) stabilizes hybridization, e.g., duplex formation; and (iv) provides a member of a binding complex or affinity set, e.g., affinity, antibody-antigen, ionic complexes, hapten-ligand (e.g., biotin-avidin). In still other embodiments, use of labels can be accomplished using any one of a large number of known techniques employing known labels, linkages, linking groups, reagents, reaction conditions, and analysis and purification methods.

miRNAs and mRNAs can be detected by direct or indirect methods. In a direct detection method, one or more miRNAs are detected by a detectable label that is linked to a nucleic acid molecule. In such methods, the miRNAs may be labeled prior to binding to the probe. Therefore, binding is detected by screening for the labeled miRNA that is bound to the probe. The probe is optionally linked to a bead in the reaction volume.

In certain embodiments, nucleic acids are detected by direct binding with a labeled probe, and the probe is subsequently detected. In one embodiment of the invention, the nucleic acids, such as amplified miRNAs, are detected using FIexMAP Microspheres (Luminex) conjugated with probes to capture the desired nucleic acids. Some methods may involve detection with polynucleotide probes modified with fluorescent labels or branched DNA (bDNA) detection.

In other embodiments, nucleic acids are detected by indirect detection methods. For example, a biotinylated probe may be combined with a streptavidin-conjugated dye to detect the bound nucleic acid. The streptavidin molecule binds a biotin label on amplified miRNA, and the bound miRNA is detected by detecting the dye molecule attached to the streptavidin molecule. In one embodiment, the streptavidin-conjugated dye molecule comprises Phycolink® Streptavidin R-Phycoerythrin (PROzyme). Other conjugated dye molecules are known to persons skilled in the art.

Labels include, but are not limited to: light-emitting, light-scattering, and light-absorbing compounds which generate or quench a detectable fluorescent, chemiluminescent, or bioluminescent signal (see, e.g., Kricka, L., Nonisotopic DNA Probe Techniques, Academic Press, San Diego (1992) and Garman A., Non-Radioactive Labeling, Academic Press (1997).). Fluorescent reporter dyes useful as labels include, but are not limited to, fluoresceins (see, e.g., U.S. Pat. Nos. 5,188,934, 6,008,379, and 6,020,481), rhodamines (see, e.g., U.S. Pat. Nos. 5,366,860, 5,847,162, 5,936,087, 6,051,719, and 6,191,278), benzophenoxazines (see, e.g., U.S. Pat. No. 6,140,500), energy-transfer fluorescent dyes, comprising pairs of donors and acceptors (see, e.g., U.S. Pat. Nos. 5,863,727; 5,800,996; and 5,945,526), and cyanines (see, e.g., WO 9745539), lissamine, phycoerythrin, Cy2, Cy3, Cy3.5, Cy5, Cy5.5, Cy7, FluorX (Amersham), Alexa 350, Alexa 430, AMCA, BODIPY 630/650, BODIPY 650/665, BODIPY-FL, BODIPY-R6G, BODIPY-TMR, BODIPY-TRX, Cascade Blue, Cy3, Cy5, 6-FAM, Fluorescein Isothiocyanate, HEX, 6-JOE, Oregon Green 488, Oregon Green 500, Oregon Green 514, Pacific Blue, REG, Rhodamine Green, Rhodamine Red, Renographin, ROX, SYPRO, TAMRA, Tetramethylrhodamine, and/or Texas Red, as well as any other fluorescent moiety capable of generating a detectable signal. Examples of fluorescein dyes include, but are not limited to, 6-carboxyfluorescein; 2′,4′,1,4,-tetrachlorofluorescein; and 2′,4′,5′,7′,1,4-hexachlorofluorescein. In certain aspects, the fluorescent label is selected from SYBR-Green, 6-carboxyfluorescein (“FAM”), TET, ROX, VICTM, and JOE. For example, in certain embodiments, labels are different fluorophores capable of emitting light at different, spectrally-resolvable wavelengths (e.g., 4-differently colored fluorophores); certain such labeled probes are known in the art and described above, and in U.S. Pat. No. 6,140,054, incorporated by reference in its entirety. A dual labeled fluorescent probe that includes a reporter fluorophore and a quencher fluorophore is used in some embodiments. It will be appreciated that pairs of fluorophores are chosen that have distinct emission spectra so that they can be easily distinguished.

In still another embodiment, labels are hybridization-stabilizing moieties which serve to enhance, stabilize, or influence hybridization of duplexes, e.g., intercalators and intercalating dyes (including, but not limited to, ethidium bromide and SYBR-Green), minor-groove binders, and cross-linking functional groups (see, e.g., Blackburn et al., eds. “DNA and RNA Structure” in Nucleic Acids in Chemistry and Biology (1996)).

In further aspects, methods relying on hybridization and/or ligation to quantify miRNAs or mRNAs may be used, including oligonucleotide ligation (OLA) methods and methods that allow a distinguishable probe that hybridizes to the target nucleic acid sequence to be separated from an unbound probe. As an example, HARP-like probes, as disclosed in U.S. Publication No. 2006/0078894, incorporated by reference in its entirety, may be used to measure the quantity of miRNAs.

In an additional embodiment of the method, a probe ligation reaction may be used to quantify miRNAs or mRNAs. In a Multiplex Ligation-dependent Probe Amplification (MLPA) technique (Schouten et al., Nucleic Acids Research 30:e57 (2002), incorporated by reference in its entirety), pairs of probes which hybridize immediately adjacent to each other on the target nucleic acid are ligated to each other only in the presence of the target nucleic acid. In some aspects, MLPA probes have flanking PCR primer binding sites. MLPA probes can only be amplified if they have been ligated, thus allowing for detection and quantification of miRNA biomarkers.

The methods described herein are useful for application to any lung cancer, for example small cell lung cancer and non-small cell lung cancer, including adenocarcinoma, squamous cell carcinoma and large-cell lung cancer.

In one embodiment, reducing MDM2 expression by administration of miR-660 inhibits tumor growth. Without wishing to be bound by theory, high expression level of MDM2 is associated with a poor prognosis. Additional embodiments of the invention further comprise measuring the expression level of MDM2 in a lung tumor tissue or biological fluid sample of the patient. In these embodiments, an MDM2 expression level in the lung tumor tissue sample or a biological fluid sample of the patient that is above an MDM2 expression level in a lung tumor tissue sample from a plurality of lung cancer patients that had a favorable prognosis indicates that the patient has a poor prognosis.

The measurement of expression of proteins such as MDM2 and p53 can be by any means known in the art. In some embodiments, the expression level of MDM2 or p53 is determined by mRNA expression, as discussed above.

Alternatively, and in further embodiments of the disclosure, gene expression of proteins such as MDM2 and p53 may be determined by analysis of expressed protein in a cell sample of interest, for example, by use of one or more binding proteins such as antibodies specific for one or more epitopes of the individual gene products (proteins), or proteolytic fragments thereof, in a cell sample or in a bodily fluid of a subject. The cell sample may be one enriched from the blood of a subject, such as by use of labeled antibodies against cell surface markers followed by fluorescence activated cell sorting (FACS). Such antibodies may be labeled to permit their detection after binding to the gene product. Detection methodologies suitable for use in the practice of the disclosure include, but are not limited to, immunohistochemistry of cell containing samples or tissue, enzyme linked immunosorbent assays (ELISAs) including antibody sandwich assays of cell containing tissues or blood samples, mass spectroscopy, and immuno-PCR.

One of the effects of the treatment methods described herein is an increase in p53. Without being bound to any particular mechanism, it is believed that the increase in p53 is the cause of the tumor suppression in this treatment.

Thus, in some embodiments, the invention methods further comprise measuring the expression level of p53 in a tumor tissue sample of the patient. In these embodiments, an absence of p53 in the tumor tissue sample indicates that the patient would not benefit from therapy that reduces MDM2 expression levels, and where p53 is present, but at an expression level below a p53 expression level in a lung tumor tissue sample from a plurality of lung cancer patients that had a favorable prognosis, indicates that the patient would benefit from therapy that reduces MDM2.

The Example below demonstrates that the concentration of miR-660 in bodily fluids of a lung cancer patient is lower than the miR-660 level is bodily fluids of control subjects that do not have lung cancer.

Thus, a method of diagnosing lung cancer in a patient is provided. The method comprises measuring the concentration of miR-660 in a bodily fluid of the patient. In this method, a miR-660 concentration in the patient below a miR-660 concentration in a plurality of control subjects that do not have lung cancer indicates that the patient has lung cancer.

As discussed above, these embodiments are not narrowly limited to any particular bodily fluid. Examples of useful bodily fluids are peripheral blood, serum, plasma, ascites, urine, sputum, saliva, broncheoalveolar lavage fluid, cyst fluid, pleural fluid, peritoneal fluid, lymph, pus, lavage fluids from sinus cavities, bronchopulmonary aspirates, and bone marrow aspirates.

In some embodiments, the concentration of miR-660 is measured by isolating total RNA from the bodily fluid, reverse transcribing the miR-660 into miR-660 cDNA, and quantifying the miR-660 cDNA using real-time PCR.

This method is useful for application to any lung cancer, for example small cell lung cancer and non-small cell lung cancer, including adenocarcinoma, squamous cell carcinoma and large-cell lung cancer.

In some embodiments, this method further comprises measuring the expression level of mouse double minute 2 (MDM2) in a lung tumor tissue sample of the patient if the patient has lung cancer. In these embodiments, an MDM2 expression level in the lung tumor tissue sample of the patient that is above an MDM2 expression level in non-cancerous tissue indicates that the patient would benefit from therapy that reduces MDM2 expression levels in the patient's lung tumor tissue.

As previously discussed, the MDM2 expression level may be determined by any means known in the art. In some embodiments, the MDM2 expression level is measured by measuring the MDM2 mRNA level. In other embodiments, the MDM2 expression level is measured by measuring the MDM2 protein level.

The example below also demonstrates that the concentration of miR-660 in cancerous lung tissue is lower than in paired normal lung tissue (see, e.g., FIG. 2B). Thus, an additional method of diagnosing lung cancer in a patient is provided. The method comprises measuring the concentration of mir-660 in (a) tissue suspected of being lung cancer in the patient and (b) normal tissue of the patient, wherein a lower level of miR-660 in the suspected tissue than in the normal tissue indicates that the suspected tissue is lung cancer.

In some embodiments of this method, the non-cancerous tissue is from the patient. In other embodiments, the non-cancerous tissue is from a control subject or a plurality of control subjects.

In various embodiments, this method further comprises measuring the expression level of p53 in a tumor tissue sample of the patient if the patient has lung cancer. In these embodiments, an absence of p53 in the tumor tissue sample indicates that the patient would not benefit from therapy that reduces MDM2 expression levels, and where p53 is present but at an expression level below a p53 expression level in a tissue sample from non-cancerous tissue indicates that the patient would benefit from therapy that reduces MDM2.

In some of these embodiments, the non-cancerous tissue is from the patient. In some of these embodiments, the normal tissue is obtained from the same specimen as the suspect tissue. In other embodiments, the non-cancerous tissue is from a control subject or a plurality of control subjects.

This method is useful for application to any lung cancer, for example small cell lung cancer and non-small cell lung cancer, including adenocarcinoma, squamous cell carcinoma and large-cell lung cancer.

The tissue for these embodiments may be obtained by any means, for example bronchoscopy, needle aspiration, core biopsy, thoracentesis, or thoracotomy. Further, any type of tissue preparation may be used, for example, fresh frozen tissue of FFPE tissue.

The present invention is further illustrated by reference to the following Examples. However, it is noted that these Examples, like the embodiments described above, are illustrative and are not to be construed as restricting the scope of the invention in any way.

### Examples

### Example

### MiR-660 is Down-Regulated in Lung Cancer Patients and its Administration Inhibits Lung Tumorigenesis by Targeting the MDM2-p53 Interaction

### Example Summary

Lung cancer represents the leading cause of cancer-related death in developed countries. Despite advances in diagnostic and therapeutic techniques, the 5-year survival rate remains very low. The research for novel therapies directed to biological targets has modified therapeutic approaches, but the frequent engagement of resistance mechanisms and the substantial costs limit the ability to reduce lung cancer mortality. MicroRNAs (miRNAs) are small non-coding RNAs with regulatory functions in controlling cancer initiation and progression. In this study we found that miR-660 expression is down-regulated in lung tumors compared with adjacent normal tissues and in plasma samples of lung cancer patients with poor prognosis, suggesting a potential functional role of this miRNA in lung tumorigenesis. Transient over-expression of miR-660 using miRNA mimics reduced migration, invasion, and proliferation properties and increased apoptosis in NCI-H460, LT73, and A549 p53 wild-type lung cancer cells. Furthermore, stable over-expression using lentiviral vectors in NCI-H460 and A549 cells inhibited tumor xenograft growth in immunodeficient mice (95% and 50% reduction compared to control, respectively), whereas the effects of miR-660 over-expression were absent in H1299, a lung cancer cell line lacking p53, in both in vitro and in vivo assays. These effects of miR-660 were mediated through mouse double minute 2 (MDM2), a key regulator of the level and function of p53 tumor suppressor protein. MDM2 is thus identified and validated as a new direct target of miR-660.

MiR-660 thus acts as a tumor suppressor miRNA, and replacement of miR-660 expression is a new therapeutic approach for p53 wild-type lung cancer treatment.

### Material and Methods

Population study. Tissue and plasma samples were collected from high-risk heavy smoker volunteers aged 50 or older, including current or former smokers with a minimum pack/year index of 20 enrolled in two independent low-dose computed tomography (LDCT) trials performed at the Istituto Nazionale dei Tumori-Istituto Europeo di Oncologia (INT-IEO) and the Multicentric Italian Lung Detection (MILD) trials (Pastorino et al., 2003; Pastorino et al., 2012). Twenty lung cancer patients from the MILD trial were selected for the analysis on tissue samples; 19 lung cancer patients and 27 disease-free individuals grouped in 5 pools from the INT-IEO trial were selected for the analysis on plasma samples (Table 1).

TABLE 1
Characteristics of enrolled subjects

| | Trial INT-IEO | | Trial ILD | |
|---|---|---|---|---|
| | (n = 18) | | (n = 20) | |
| Gender | | | | |
| Male | 12 | (66.7%) | 15 | (75.0%) |
| Female | 6 | (33.3%) | 5 | (25%) |
| Age (years) | 58.4 ± 5.4 | | 61.7 ± 6.6 | |
| Smoking habit (Pack-Year index) | 60.8 ± 23.2 | | 55 ± 19.8 | |
| Histotype | | | | |
| ADC | 14 | (77.8%) | 14 | (70.0%) |
| SCC | 3 | (16.7%) | 5 | (25.0%) |
| other | 1 | (5.5%) | 1 | (5.0%) |
| Stage | | | | |
| Ia-Ib | 11 | (61.1%) | 14 | (70.0%) |
| II-III-IV | 7 | (38.9%) | 6 | (30.0%) |
| Median Follow up (months) | 66 | | 26 | |
| Prognosis | | | | |
| Disease free | 10 | (55.6%) | 14 | (70.0%) |
| Alive with disease | 0 | | 1 | (5.0%) |
| Dead | 8 | (44.4%) | 5 | (25.0%) |

MiRNA expression analysis. For plasma samples, total RNA was isolated from 200 μl of plasma using the mirVana™ PARIS kit (Life Technologies/Thermo Fisher Scientific) according to the manufacturer's instructions and eluted in 50 μl of Elution Buffer. High-throughput analyses were performed using the Megaplex™ Pools Protocol on microfluidic card type A (Life Technologies/Thermo Fisher Scientific) as previously described (Boeri et al., 2011).

For tissue samples, total RNA was extracted using Trizol® (Invitrogen/Thermo Fisher Scientific) following manufacturer's instructions and quantified using the NanoDrop 2000 (Thermo Scientific).

For cultured cells, total RNA was isolated using mirVana PARIS Kit (Life Technologies/Thermo Fisher Scientific) following the manufacturer's instructions. Reverse transcription was performed using the TaqMan® microRNA Reverse Transcription Kit according to the manufacturer's instruction (Applied Biosystems/Thermo Fisher Scientific). MiRNA expression was analyzed by the Applied Biosystems 7900 System and normalized to the small nucleolar RNU6B for tissues and RNU48 for cells.

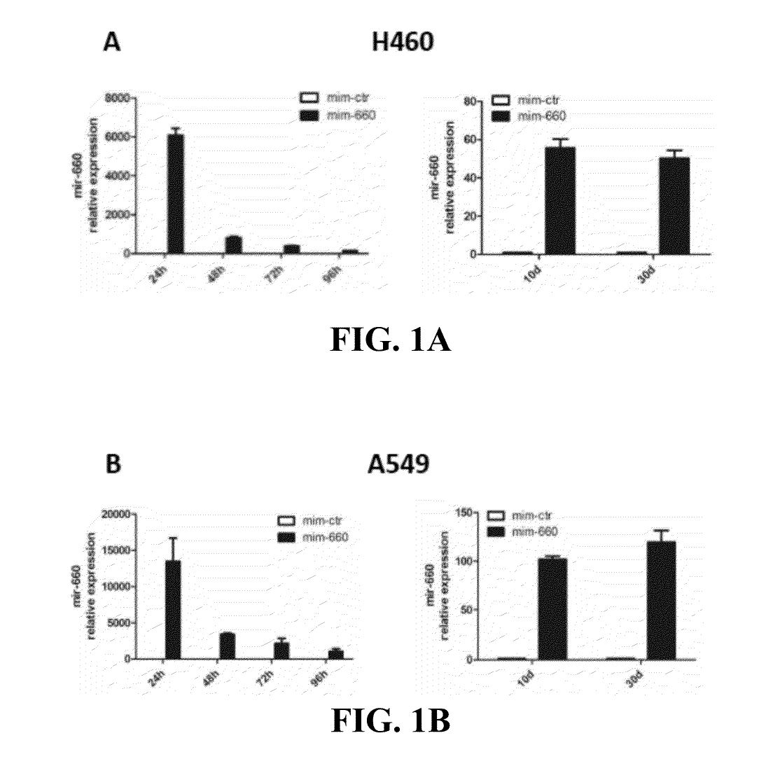

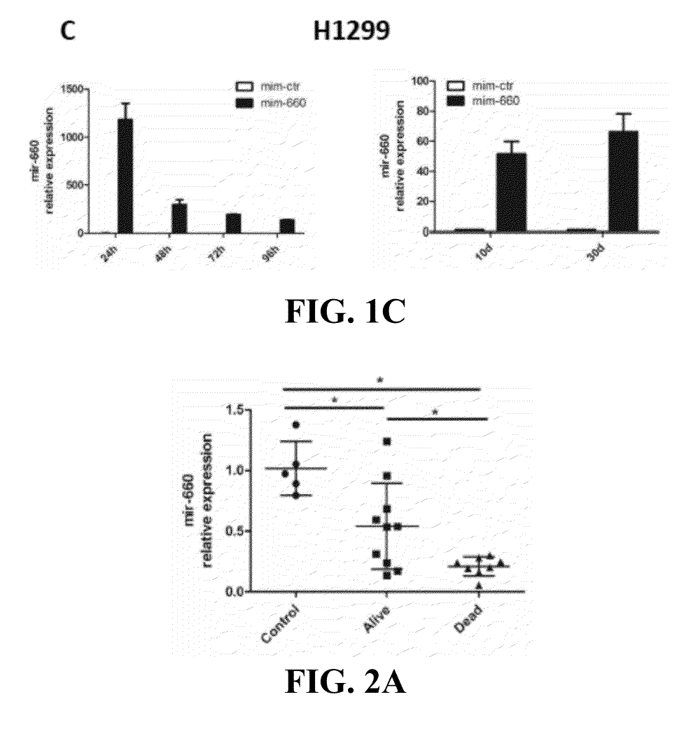

Cell lines and miRNA transfection. Human lung cancer cell lines, H460, LT73, A549 and H1299, were obtained from the American Type Culture Collection (ATCC). LT73 cells were derived in our laboratory from a primary lung tumor of a 68-year old Caucasian male with lung adenocarcinoma. Cells were cultured in RPMI 1640 medium supplemented with 10% heat inactivated fetal bovine serum (FBS) and 1% penicillin-streptomycin (Sigma-Aldrich). Cells were transfected using mirVana miRNA mimics using Lipofectamine® 2000 (Invitrogen/Thermo Fisher Scientific) according to the manufacturer's instructions (FIGS. 1A-1C).

Proliferation assay. For proliferation assays, cells were seeded in a 12-well plate at 2×10⁵ cells for A549, H1299 and LT73 and 1.5×10⁵ cells for H460. Viable cells were counted after 72 and 120 hours by Trypan blue staining Each experiment was performed in triplicate.

Migration and invasion assay. For migration assays, 10⁵ cells were plated on the top chamber of FluoroBlok Cell Culture Inserts (BD Biosciences). RPMI plus 10% FBS was added to the bottom chamber and incubated at 37° C. and 5% CO₂. For the invasion assay FluoroBlok Cell Culture Inserts were coated with matrigel (BD Biosciences). After 24 hours, cells that had migrated to the bottom side of the insert were fixed and stained with DAPI. Migrated cells were counted using fluorescence microscopy. Migration and invasion data are expressed as the number of migrated miR-660 over-expressing cells vs. the number of migrated control cells.

Apoptosis evaluation. Apoptosis was measured by quantifying the percentage of Annexin V^{pos}/Propidium Iodide^{neg} cells by flow cytometry. The percentage of apoptotic cells was evaluated 48 hours after miRNA transfection using the Annexin V-FITC Kit (Miltenyi Biotec) according to the manufacturer's protocol.

Cell cycle evaluation. Cells were fixed with 70% cold ethanol and stained with propidium iodide (50 μg/ml) for 40 minutes. Cells were analyzed by flow cytometry using BD FACS Calibur and Cell Quest software (BD Biosciences).

Western Blot analysis. Proteins were extracted by incubation with RIPA buffer and quantified by Bradford reagent. Twenty-five micrograms of protein were separated on Nupage 4-12% polyacrylamide gels (Invitrogen/Thermo Fisher Scientific) and transferred to polyvinylidene difluoride membranes (PVDF, GE Healthcare) to be probed with the following antibodies: mouse anti-MDM2 (1:500, Abcam) and rabbit anti-β-actin (1:5000, Sigma). For detection, goat anti-rabbit or goat anti-mouse secondary antibodies conjugated to horseradish peroxidase (1:2000, GE Healthcare) were used. Signal detection was performed via chemiluminescence reaction (ECL, GE Healthcare).

p53 ELISA. P53 protein levels in cancer cells lysates were measured using a p53 Human ELISA kit (Abcam) according to manufacturer's instructions.

Luciferase assays. To investigate whether MDM2 is a direct target of mir-660, the 3′ untranslated region (UTR) of MDM2 was purchased from Switchgear Genomics. Conserved binding sites in MDM2 3′UTR at position 3333-3340 was identified using TargetScan (http://www.targetscan.org). An empty vector was used as control. Furthermore, the predicted target site for miR-660 was mutated by direct mutagenesis of the pLightSwitch_MDM2 3′UTR vector, using the PCR-based QuikChange II XL site-directed mutagenesis kit (Stratagene, La Jolla, Calif.) according to the manufacturer's instructions and the following primers: Fw 5′-CAAAACCACTTTTACCAAATACAGAGTTAAATTTG-3′ (SEQ ID NO: 9); Rev 5′-CAAATTTAACTCTGTATTTGGTAAAAGTGGTTTTG-3′ (SEQ ID NO: 10). The presence of the mutations was confirmed by sequencing. The different luciferase constructs were transfected into HEK293 cells together with miR-660 or a scrambled oligonucleotide sequence (control). Cells were cultured for 48 hours and assayed with the Luciferase Reporter Assay System (Switchgear Genomics).

Generation of stable miR-660 over-expressing cells. To obtain stable miR-660 over-expressing cells experiments were performed using the SMARTchoice lentiviral vector (Thermo Fisher Scientific). Lung cancer cells were seeded at 5×10⁴ in each well of 24-well plates and infected with miR-660 or control lentiviral vector at the multiplicity of infection (m.o.i) of 10 (10 infectious units for each target cell). After 72 hours, cells were selected with puromycin, and miRNA over-expression was quantified at 10 and 30 days post infection (FIGS. 1A-1C).

In vivo assays. Animal studies were performed according to the Ethics Committee for Animal Experimentation of the Fondazione IRCCS Istituto Nazionale Tumori, according to institutional guidelines previously described (Workman et al., 2010). All experiments were carried out with female CD-1 nude mice or SCID mice, 7-10 weeks old (Charles River Laboratories). Mice were maintained in laminar flow rooms, with constant temperature and humidity and had free access to food and water.

Lung cancer cells, transfected with mimic-660 or control, were harvested and resuspended in Matrigel/RPMI (1:1). 5×10⁵ cells were injected subcutaneously in the flanks of 4 to 6 weeks old female nude mice. For each group, 5 mice were used and injections were performed in two flanks of each animal (n=10 tumors/group). Xenograft growth was measured weekly using a caliper.

Statistical Analysis. Statistical significance was determined with unpaired or paired t tests. P-values less than 0.05 were considered statistically significant.

### Results

Diagnostic and prognostic value of miR-660 in plasma and tissue samples of lung cancer patients. In order to identify miRNAs differentially expressed between 18 lung cancer patients and 27 matched disease-free individuals grouped in five pools (Table 1) we first performed high-throughput miRNA expression profile of plasma samples collected during the INT-IEO LDCT screening trial (Pastorino et al., 2003). Among those miRNAs significantly deregulated between patients and controls, we found that miR-660 was progressively down-modulated in patients with favorable prognosis (alive) (miR-660 relative expression=0.54±0.35 vs. 1.02±0.22, p<0.05) and patients with poor prognosis (dead) (miR-660 relative expression=0.21±0.08 vs. 1.02±0.22, p<0.05) (FIG. 2A) compared to controls.

Twenty paired tumor and normal lung tissues obtained from lung cancer patients enrolled in the MILD trial (Pastorino et al., 2012) were selected (Table 1) to determine miR-660 expression in those tissues. As shown in FIG. 2B, miR-660 was significantly reduced in tumor compared to paired normal lung tissue (miR-660 relative expression=0.38±0.2 vs. 1.21±0.85, p<0.05). P53 mutational status was analyzed in the series of lung cancer patients used for tissue microRNA profiling. P53 mutations were found in 9 out of 20 patients, but the p53 status did not correlate (p=0.37) with mir-660 expression levels.

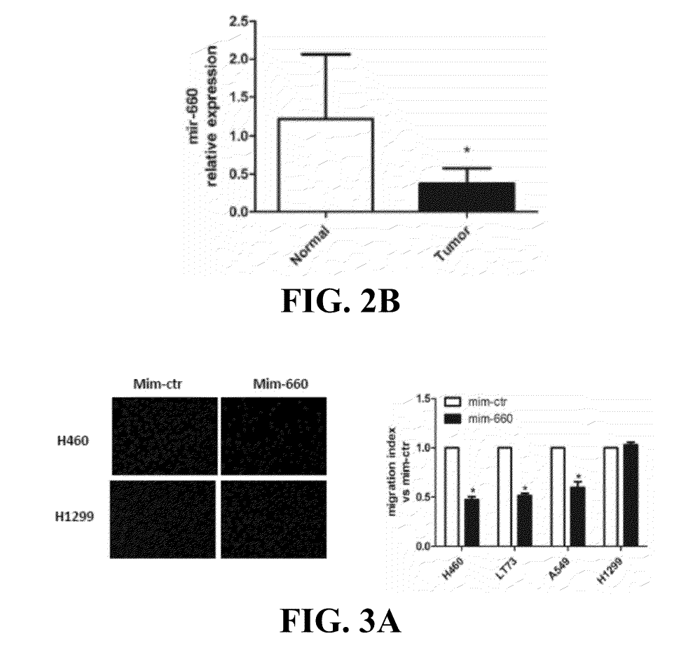

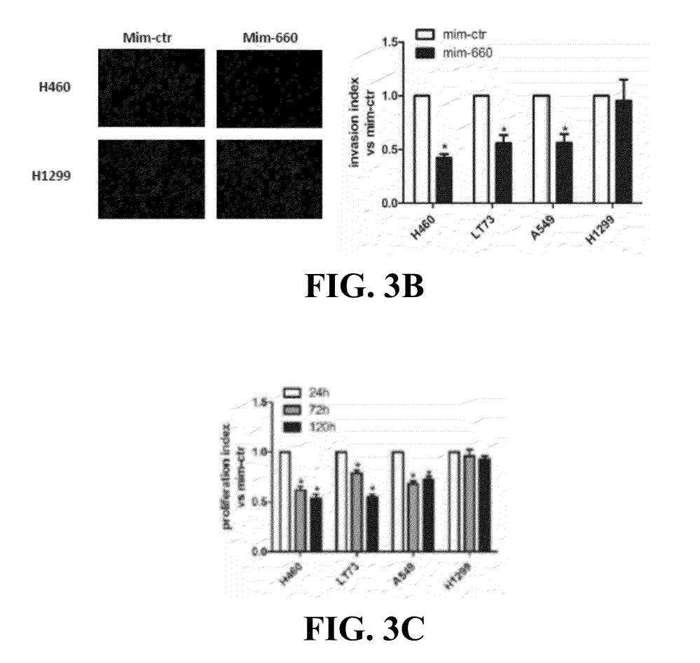

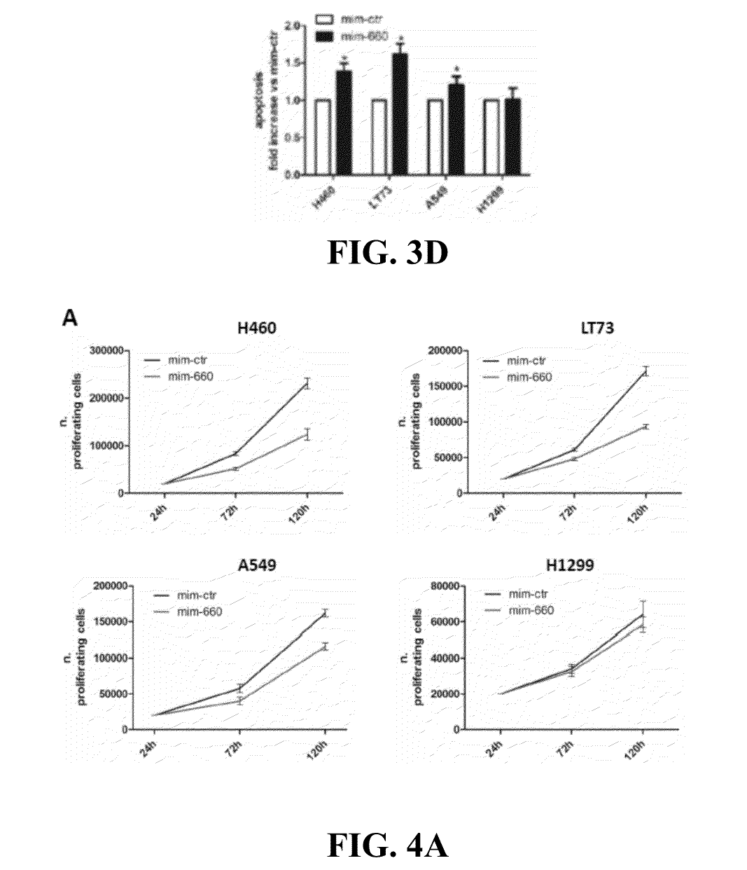

MiR-660 reduces cancer cell functionality. In order to understand the functional role of miR-660 in lung tumorigenesis, a series of in vitro experiments was performed using commercially available miRNA mimics on four different lung cancer cell lines (H460, LT73, A549 and H1299). MiR-660 over-expression resulted in a significant decrease of migratory (FIG. 3A) and invasive (FIG. 3B) capacity of the three p53 wild type cancer cell lines, but not of the H1299 lung cancer cell line, which lacks functional p53. Furthermore, miR-660 led to a reduction in cell proliferation at 72 and 120 hours after mir-660 transfection was detected in p53 wild-type cells only (FIG. 4A). To explain the decrease in cell proliferation, apoptosis was evaluated by flow cytometry by measuring the AnnexinV^{pos}/PI^{neg} cells in mir-660 transfected cell lines and observed a 20-60% increase in the number of apoptotic cells after 48 hours compared to cells transfected with mimic control (FIG. 4B).

Cell cycle progression was also evaluated after transient transfection of miR-660. In those studies, there was a significant increase of G0/G1 cells indicating a cell cycle arrest (Table 2). Interestingly, the absence of the effects observed in in vitro experiments in H1299 suggested a potential involvement of miR-660 in targeting the p53 pathway.

TABLE 2
MiR-660 over-expression induced G0/G1 cell cycle arrest

| | | % G0/G1 | | % S | % G2/M |
|---|---|---|---|---|---|
| | | cells | p-value | cells | cells |
| NCI-H460 | Mim-ctr | 77.9 ± 1.5 | <0.01 | 12.1 ± 1.6 | 7.4 ± 2.0 |
| | Mim-660 | 83.8 ± 1.0 | | 10.8 ± 1.0 | 3.0 ± 1.3 |
| LT73 | Mim-ctr | 66.1 ± 1.7 | 0.01 | 16.7 ± 3.8 | 15.6 ± 1.6 |
| | Mim-660 | 72.7 ± 1.7 | | 14.1 ± 1.0 | 12.0 ± 1.3 |
| A549 | Mim-ctr | 56.5 ± 3.2 | 0.03 | 19.9 ± 1.3 | 15.0 ± 3.0 |
| | Mim-660 | 62.6 ± 2.3 | | 18.8 ± 1.2 | 12.9 ± 3.2 |
| H1299 | Mim-ctr | 81.9 ± 5 | 0.40 | 8.2 ± 3.2 | 6.1 ± 2.5 |
| | Mim-660 | 83.8 ± 3.1 | | 7.5 ± 1.3 | 5.4 ± 1 |

All data are expressed as mean ± SEM.
(n = 5, *p < 0.05 vs. mim-ctr)

MDM2 is a Direct Target of miR-660.

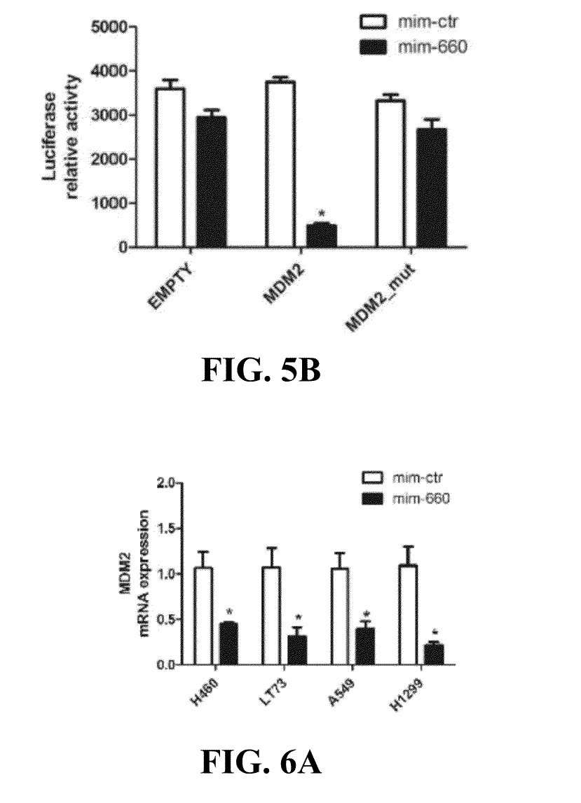

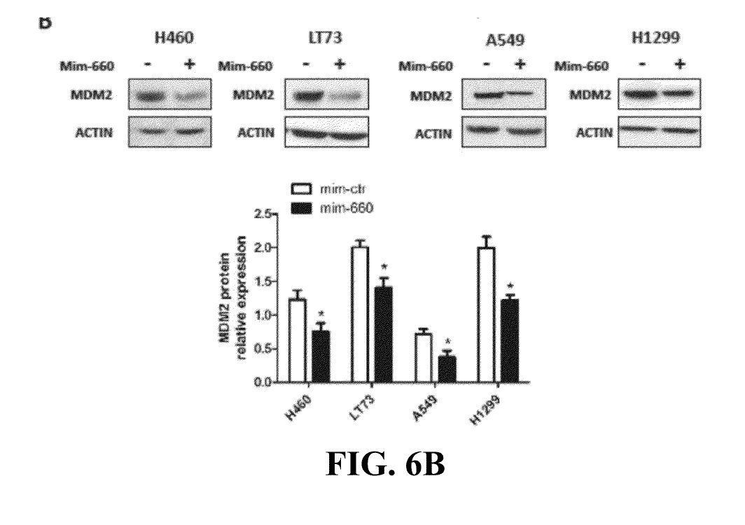

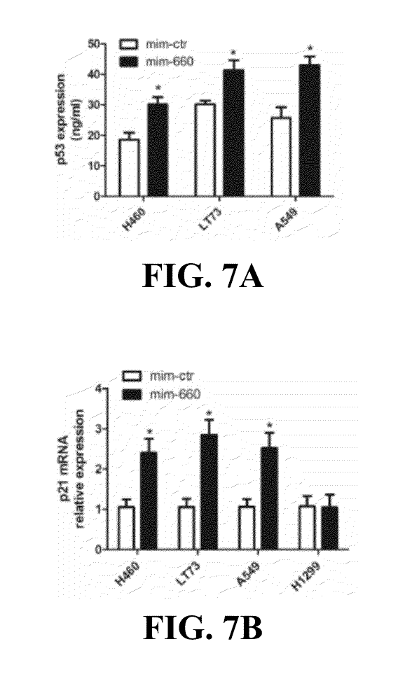

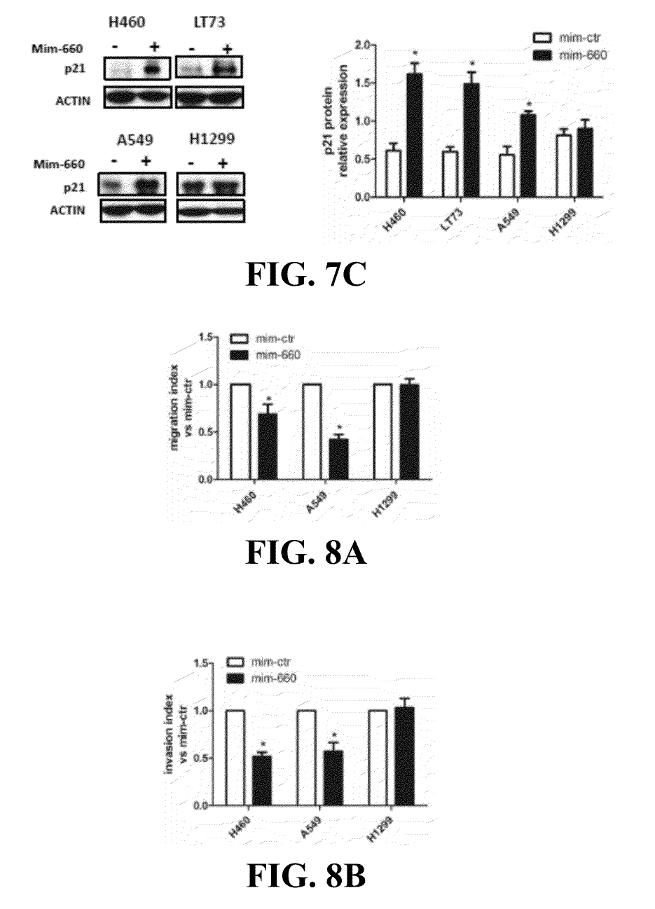

Using in silico programs we first identified putative miR-660 targets, focusing on those mRNA encoding for proteins that are components of the p53 pathway. The analysis identified the 3′ UTR of MDM2 as a complementary sequence for the binding of miR-660 (FIG. 5A). MDM2 is an E3 ligase and its role is the physiological regulation of p53. To prove that MDM2 is a direct target of miR-660, a luciferase reporter assay was performed using commercial custom made 3′ UTR MDM2. There was a strong down-modulation (87% reduction) of the luciferase activity when co-transfected with miR-660 (FIG. 5B). Target specificity was verified using a 3′UTR EMPTY vector and also by site-directed mutagenesis in the putative miR-660 minding sites, where no change in luciferase activity was observed (FIG. 5B). There was also a significant reduction of MDM2 mRNA 72 hours after miR-660 transfection, as measured by Real-Time PCR (% MDM2 mRNA reduction: 60% for NCI-H460; 70% for LT73 and 63% for A549 compared to control) (FIG. 6A), as well as a reduction of MDM2 protein expression, as determined by Western Blot in all tested cell lines (% MDM2 protein reduction: 39% for NCI-H460; 30% for LT73 and 47% for A549 compared to control) (FIG. 6B). Furthermore, to confirm that the effects of mir-660 replacement were p53-dependent, total p53 levels on cell lysates showed a significant increase in p53 protein expression in all cell lines (p53 protein increase: 63% for NCI-H460; 37% for LT73 and 67% for A549 compared to control) (FIG. 7A).

To demonstrate that the antitumoral activity of mir-660 is p53-dependent, mRNA levels of p21^{WAF1/CIP1}, a cyclin-dependent kinase inhibitor which functions as p53-dependent cell cycle checkpoint, were analyzed and a significant increase of p21 levels after mir-660 over-expression (2.3 fold increase in NCI-H460; 2.7 in LT73 and 2.4 in A549 compared to control) were observed (FIG. 7B). Accordingly, to confirm p21^{WAF1/CIP1} mRNA level up-regulation, a western blot analysis on cell lysates showed a comparable increase of p21^{WAF1/CIP1} protein levels (2.6 fold increase in NCI-H460; 2.5 in LT73 and 1.7 in A549 compared to control) (FIG. 7C).

Interestingly, we observed MDM2 down-modulation was also viable in H1299 p53-null cells (% MDM2 reduction: 40%) without stimulation of p21^{WAF1/CIP1} transcription or protein expression, indicating that the presence of a functional p53 protein is fundamental for miR-660 antitumoral effects through the regulation of MDM2 levels.

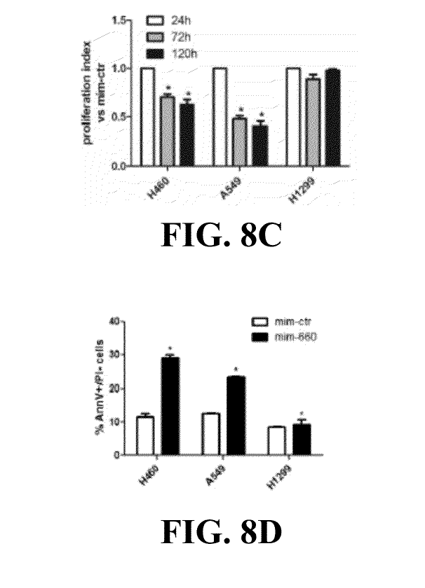

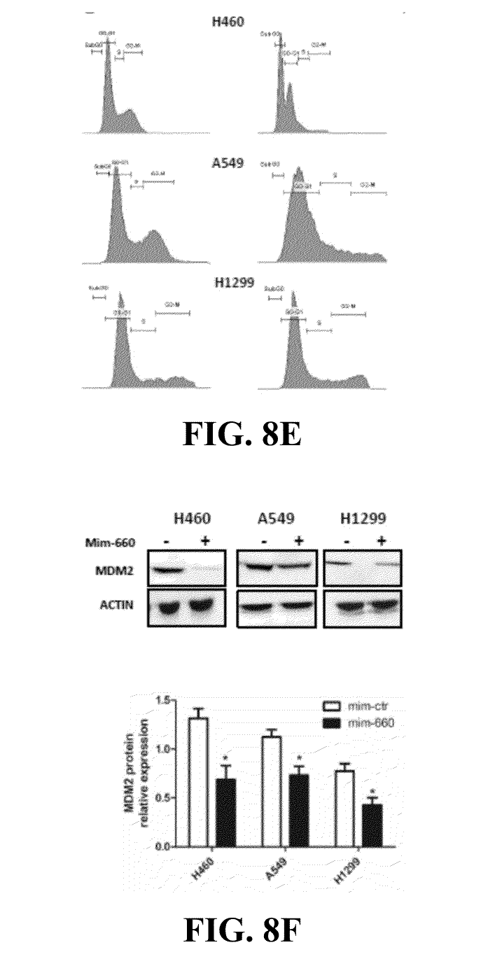

MiR-660 stable over-expression has tumor suppressive effects in vitro. In order to obtain a stable mir-660 over-expression in all cell lines used, stable mir-660 transfectants were created using lentiviral vectors (FIGS. 1A-1C). Furthermore, to confirm mir-660 antitumoral activity, in vitro assays were performed using stable mir-660 over-expressing cells and we observed a decrease in migratory (FIG. 8A) and invasive (FIG. 8B) ability of these cells and a reduction in cell proliferation compared to control (FIG. 8C). Stable mir-660 over-expression induced a significant increase of apoptotic cells measured as the AnnexinV^{pos}/PI^{neg} in NCI-H460 and A549 cells (2.5 fold increase in NCI-H460 and 1.8 in A549 compared to control) (FIG. 8D). According to data obtained with transient transfection, in H1299 cells these effects were totally abrogated. Interestingly, cell cycle analysis showed a marked increase of apoptotic cells (subG0) and a strong G0/G1 arrest in NCI-H460 and A549 respectively, whereas no differences were observed in H1299 p53-null cells (FIG. 8E and TABLE 3). In all cell lines, stable mir-660 over-expression reduced MDM2 protein levels as shown by Western Blot analysis (48% protein reduction in NCI-H460; 35% in A549 and 45% in H1299 compared to control) (FIG. 8F). Stable mir-660 transfectants of LT73 cells could not be obtained likely due to the toxicity of GFP reporter gene in this primary established cell line.

TABLE 3
Stable MiR-660 over-expression impaired cell cycle in p53 wt cells

| | | % G0/G1 | | % G2/M | |
|---|---|---|---|---|---|
| | | cells | % S cells | cells | subG0 |
| NCI-460 | Ctr | 61.6 ± 0.4 | 24.5 ± 0.2 | 9.3 ± 1.7 | 4.5 ± 1.5 |
| | 660 | 36.4 ± 0.2 | 7.9 ± 0.2 | 3.4 ± 0.1 | 52.4 ± 0.3 |
| A549 | Ctr | 50.6 ± 0.3 | 23.3 ± 1.2 | 25.9 ± 0.9 | 0.2 ± 0.06 |
| | 660 | 72.5 ± 2.9 | 16.5 ± 1.1 | 10.6 ± 1.8 | 0.3 ± 0.2 |
| H1299 | Ctr | 72.9 ± 1.5 | 19.1 ± 1.8 | 6.1 ± 0.4 | 1.9 ± 1.6 |
| | 660 | 76.7 ± 2.0 | 15.6 ± 0.8 | 7.1 ± 1.2 | 0.6 ± 0.3 |

All data are expressed as mean ± SEM.
(n = 3, *p < 0.05 vs. mim-ctr)

MiR-660 inhibits xenograft tumor growth. Supported by the results showing miR-660 down-regulation in lung cancer patient tissue and plasma, and the antitumoral effects of miR-660 over-expression in in vitro assays, the potential role of this miRNA in the inhibition of tumor growth in immunodeficient mice was evaluated.

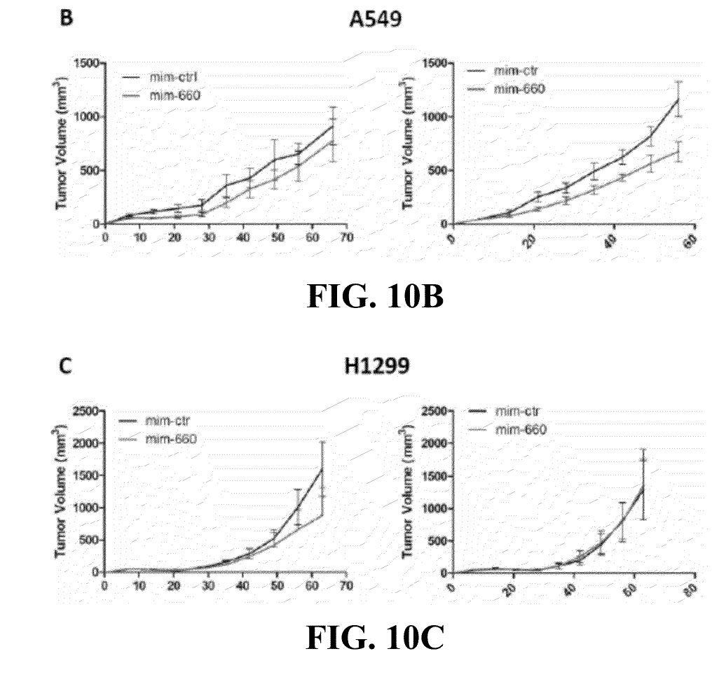

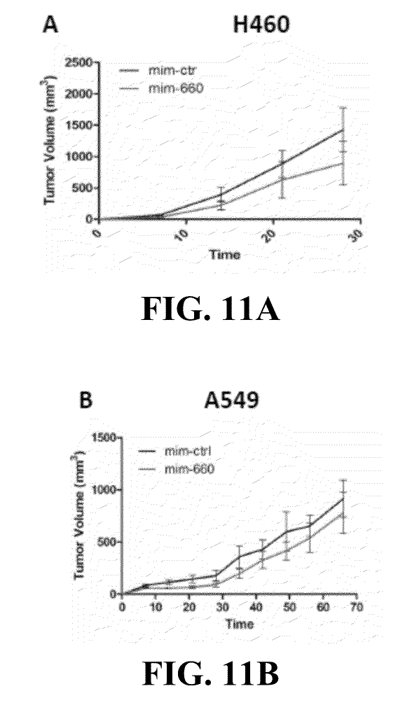

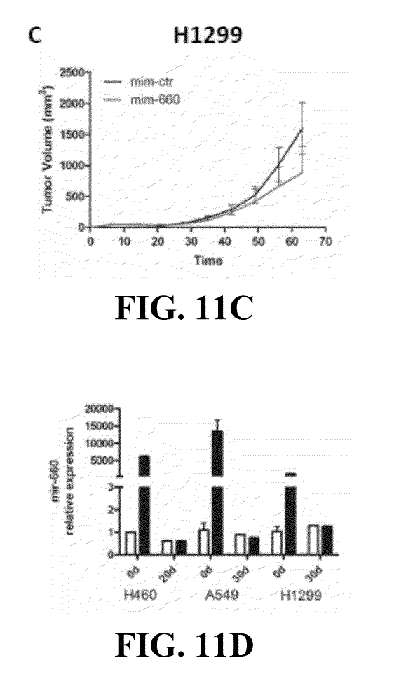

Subcutaneous injection of miR-660 transiently transfected p53 wild type NIH-H460 (FIGS. 10A and 11A) and A549 (FIGS. 10B and 11B) cells in nude mice resulted in a slight initial delay in tumor growth. After this initial effect (10-15 days for H460 and 30-35 days for A549), tumors grew with rates comparable to control transfected cells. On the other hand, in p53-null H1299 cells (FIGS. 10C and 11C), transfection of miR-660 had no effects on xenograft growth.

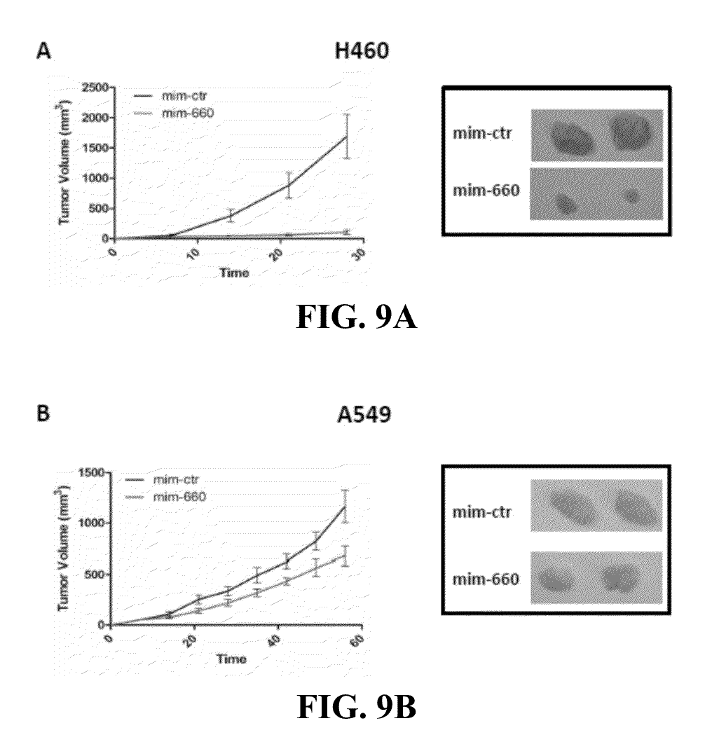

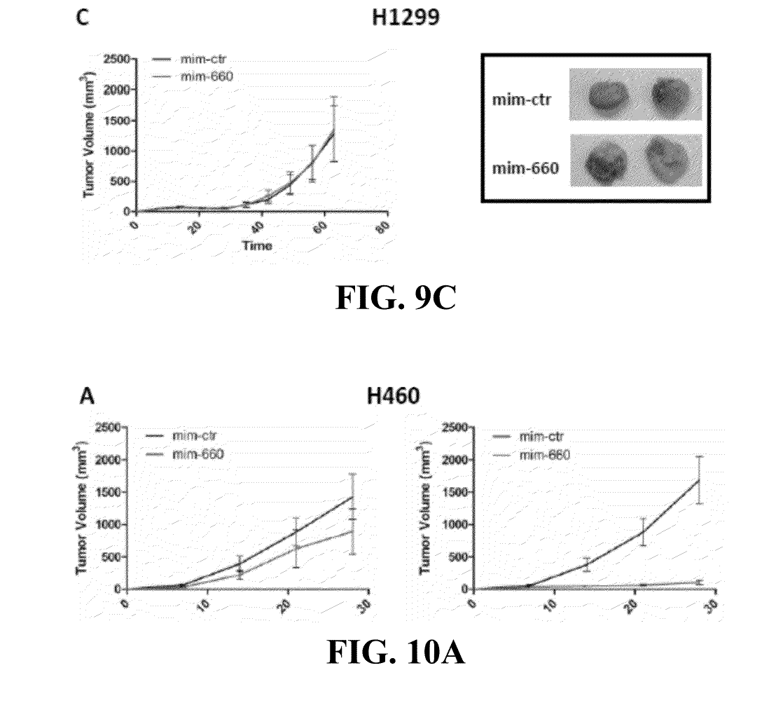

Twenty days after transient transfection, miR-660 expression levels were similar to those of control cells (FIG. 11D), suggesting a correlation between miR-660 transient over-expression and the initial delay in tumor growth observed in p53 wt cell lines xenografts. Indeed, stable transfection with miR-660 led to a complete in vivo growth inhibition (95% of reduction compared to control) in H460 cells (p53 wild type) (FIG. 9A). These effects were less pronounced in A549 (50% inhibition) and completely absent in H1299 transfected cells (FIGS. 9B-9C), lacking the MDM2 negative regulators p14^{arf} (Xie et al., 2005) and p53 protein, respectively. These results highlight the central role of the MDM2/p53 pathway in miR-660 mediated effects, including in in vivo xenograft models. Concerning LT73, cells transiently transfected were injected in immunodeficient mice and show a delay at 30-35 days compared to control (data not shown) but stable mir-660 transfectants could not be obtained likely due to the toxicity of GFP reporter gene in this primary established cell line.

### Discussion

Despite considerable efforts to improve outcomes for patients with NSCLC, the 5-year overall survival remains around 15% with a minimal improvement over the last 30 years. Surgical resection and chemotherapy are the most common treatments for lung cancer management, but in the last decade new targeted therapies directed to specific genetic alterations such as EGFR mutations or ALK translocation has led to positive results in clinical trials (Socinski et al., 2013; Blackhall et al., 2013). However, these tumors account only for the 20% of NSCLC patients (Gainor and Shaw, 2013) so new research efforts have to be directed to identify new alternative markers for targeted therapy.

MiRNAs have previously been shown to be involved in the pathogenesis of lung diseases, including lung cancer, by negatively regulating gene and protein expression by acting as oncogenes or tumor suppressors. The rationale of using miRNA as therapeutics agents in lung cancer management is based on two observations: one is that miRNAs play an important role in lung development (Sozzi et al., 2011) and their expression levels are deregulated in lung cancer patients compared to healthy subjects (Yanaihara et al., 2006). The second observation is that modulation of miRNA expression, both in vitro and in vivo, can modify cancer phenotypes (Du et al., 2009; Peng et al., 2013).

The present study demonstrates that miR-660 is down-modulated in plasma of lung cancer patients and inversely correlated with prognosis. Furthermore, we observed that miR-660 was significantly down-regulated in lung cancers compared to normal tissues, leading to the finding that miR-660 plays a functional role in lung tumorigenesis. By administering a vector that expresses miR-660, tumor growth inhibition was achieved both in vitro and in vivo, apparently mediated by miR-660-induced impairment of the MDM2/p53 interaction.

The transcription factor p53 is expressed at low concentrations in normal cells, where it plays an important role in cell cycle regulation (Leonard et al., 1995). Under physiological conditions, p53 levels are suppressed by the activity of MDM2. Disruption of the p53-MDM2 interaction is the pivotal event for p53 activation, leading to p53 stabilization and its biological functions, such as cell growth control, apoptosis, and modulation of cell migration (Roger et al., 2006; Vousden, 2000).

The over-expression of miR-660 also decreased the migratory and invasive capacity of lung cancer cells. This effect was absent in H1299, a p53-null cell line, suggesting a potential role of p53 in controlling tumor migration and invasion. P53 regulates cell migration through the modulation of cell morphology. In particular, p53 prevents filopodia formation through p38 MAPK activation (Gadea et al., 2004), and deregulates the actin cytoskeleton organization. Another potential mechanism of p53 inhibition of tumor cell motility is the inhibition of spreading and polarization of the migrating cells (Gadea et al., 2004). These studies also show that miR-660 overexpression leads to a block of proliferation in the G0/G1 checkpoint, and induction of apoptosis in a p53 dependent manner. These effects were achieved by in vitro replacement of miR-660 in p53 wild type NIH-H460 and A549 cells, whereas in H1299 p53-null cells no effects were appreciable on cell cycle or on apoptosis even if a decrease of MDM2 expression levels is observed. Similar results were obtained in in vivo experiments where a significant inhibition of tumor xenograft growth was obtained with miR-660 stable transfection in NIH-H460 cells and not with miR-660 stable transfection in p53-null H1299 cells. The hypothesis that miR-660 inhibition of tumor growth is mediated by its effects on MDM2 expression and consequently on its impact on p53 pathway is further confirmed by the mild effect on tumor xenograft growth of miR-660 stable transfection in A549, a cell line with an impairment in the p53 pathway due to the loss of p14arf, an inhibitor of MDM2 resulting in hyper-activation of this protein (Wang et al., 2005).

P53 tumor suppressor activity is frequently inactivated by mutations in NSCLC patients (Herbst et al. (2008). N Engl J Med 359, pp. 1367-1380; Yokota and Kohno, 2004) or by MDM2 which eliminates wild-type p53 (Freedman et al., 1999). MDM2 was found to be amplified in a portion of human cancers (Higashiyama et al., 1997) and in tumors that retain wild-type p53, inhibitors of MDM2 may have therapeutic value by inducing p53-dependent cytostasis or apoptosis (Bottger et al., 1997).

Upon mir-660 replacement, both in transient or in stable transfections, a tumor growth inhibition effect was shown, in vitro and in vivo, likely mediated by mir-660-induced impairment of the MDM2/p53 interaction. The transcription factor p53 is expressed at low concentration in normal cells and it plays an important role in cell cycle regulation. In physiological condition, p53 levels are suppressed by the activity of MDM2. Disruption of the p53-MDM2 interaction is the pivotal event for p53 activation, leading to p53 stabilization and its biological functions, such as cell growth control, apoptosis, and modulation of cell migration.

MiR-660 overexpression led to arrest of proliferation in G0/G1 checkpoint and induction of apoptosis in a p53-dependent manner. Indeed, these effects were achieved by in vitro replacement of mir-660 in p53 wild type NCI-H460 and A549 cells whereas in H1299 p53-null cells no effects were appreciable on cell cycle or on apoptosis even if a decrease of MDM2 expression levels was detected. It was also shown that mir-660 induced p53 stabilization and increased its transcriptional activity resulting in an up-regulation of its target gene, p21^{WAF1/CIP1}, which regulates cell cycle through inhibition of cyclin-dependent kinases required for progression from G1 to S phase and it is also involved in the apoptotic process.

Similar results were obtained in in vivo experiments where a significant inhibition of tumor xenograft growth was obtained with mir-660 stable transfection of NCI-H460 and A549 cells and not of p53-null H1299 cells.

Several studies indicate that p53 tumor suppressor activity is frequently inactivated in NSCLC patients by mutations (53% of all lung cancer) or by interaction with MDM2 which eliminates wild-type p53. MDM2 amplification occurs in 7% of human tumors with varying degrees of amplification between tumor types such as liposarcoma (50-90%), osteosarcomas (16%), esophageal carcinomas (13%), and NSCLC (6%). Notably, MDM2 amplification and p53 mutations are essentially mutually exclusive and, in the past few years, small-molecule antagonists of p53-MDM2 interaction as nutlins or MDM2 inhibitors have been developed.

These observations suggest that reconstitution of p53-dependent pathways in tumor cells is an effective therapeutic strategy and restoration of p53 activity using mir-660 represents an attractive approach for lung cancer therapy. The principal advantage of using miRNAs as therapeutic agent is that they could target several genes of redundant pathways and thus potentially able to achieve a broad silencing of pro-tumoral pathways. A very preliminary bioinformatic analysis revealed that mir-660 potentially targets several transcription factors, proteases and other regulators of cell growth and survival. Interestingly, we showed that relatively small changes in the expression of miRNA and its target gene could induce relevant phenotypic alterations of lung cancer cells, both in vitro and in vivo.

These results provide evidence that mir-660 behaves as a tumor suppressor miRNA in lung cancer and that mir-660 replacement could represent a potential nontoxic successful therapy for la large subset of lung cancer patients where p53 locus is not genetically altered by mutation or deletion.

In view of the above, it will be seen that several objectives of the invention are achieved and other advantages attained.

As various changes could be made in the above methods and compositions without departing from the scope of the invention, it is intended that all matter contained in the above description and shown in the accompanying drawings shall be interpreted as illustrative and not in a limiting sense.

All references cited in this specification are hereby incorporated by reference. The discussion of the references herein is intended merely to summarize the assertions made by the authors and no admission is made that any reference constitutes prior art. Applicants reserve the right to challenge the accuracy and pertinence of the cited references.

The following publications are incorporated by reference herein in their entireties for all purposes.
Allen J G, et al. (2009) Discovery and optimization of chromenotriazolopyrimidines as potent inhibitors of the mouse double minute 2-tumor protein 53 protein-protein interaction. J Med Chem 52(22):7044-7053.
Bartel D P (2004) MicroRNAs: genomics, biogenesis, mechanism, and function. Cell 116(2):281-297.
Bartel D P (2009) MicroRNAs: target recognition and regulatory functions. Cell 136(2):215-233.
Bishop J A, et al. (2010) Accurate classification of non-small cell lung carcinoma using a novel microRNA-based approach. Clin Cancer Res 16(2):610-619.
Blackhall F, Thatcher N, Booton R, & Kerr K (2013) The impact on the multidisciplinary team of molecular profiling for personalized therapy in non-small cell lung cancer. Lung Cancer 79(2):101-103.
Boeri M, et al. (2011) MicroRNA signatures in tissues and plasma predict development and prognosis of computed tomography detected lung cancer. Proc Natl Acad Sci USA 108(9):3713-3718.
Bottger A, et al. (1997) Design of a synthetic Mdm2-binding mini protein that activates the p53 response in vivo. Curr. Biol. 7:860-869.
Calin G A, et al. (2005) A MicroRNA signature associated with prognosis and progression in chronic lymphocytic leukemia. N Engl J Med 353(17):1793-1801.
Capoulade C, et al. (1998) Overexpression of MDM2, due to enhanced translation, results in inactivation of wild-type p53 in Burkitt's lymphoma cells. Oncogene 16(12):1603-1610.
Chen J, Wu X, Lin J, & Levine A J (1996) mdm-2 inhibits the G1 arrest and apoptosis functions of the p53 tumor suppressor protein. Mol Cell Biol 16(5):2445-2452.
Chen L, et al. (1999) Ubiquitous induction of p53 in tumor cells by antisense inhibition of MDM2 expression. Mol Med 5:21-34.
Chene P (2003) Inhibiting the p53-MDM2 interaction: an important target for cancer therapy. Nat Rev Cancer 3(2):102-109.
Croce C M (2009) Causes and consequences of microRNA dysregulation in cancer. Nat Rev Genet 10(10):704-714.
Cimmino A, et al. (2005) miR-15 and miR-16 induce apoptosis by targeting BCL2. Proc Natl Acad Sci USA 102(39):13944-13949.
De Guire V et al. (2013) Circulating miRNAs as sensitive and specific biomarkers for the diagnosis and monitoring of human diseases: promises and challenges. Clin Biochem 46:846-860.
Dmitriev P, et al. (2013a) Simultaneous miRNA and mRNA transcriptome profiling of human myoblasts reveals a novel set of myogenic differentiation-associated miRNAs and their target genes. BMC Genomics 14:265.
Dmitriev P, et al. (2013b) Defective regulation of microRNA target genes in myoblasts from facioscapulohumeral dystrophy patients. J Biol Chem 288(49):34989-35002.
Du, L. et al. (2009) miR-93, miR-98, and miR-197 Regulate Expression of Tumor Suppressor Gene FUS1. Mol Cancer Res 7:1234-1243.
Emmrich S, et al. (2012) miRNAs can increase the efficiency of ex vivo platelet generation. Ann Hematol 91(11):1673-1684.
Ettinger D S, et al. (2012) Non-small cell lung cancer. J Natl Compr Canc Netw 10(10):1236-1271.
Feng J, et al. (2004) Stabilization of Mdm2 via decreased ubiquitination is mediated by protein kinase B/Akt-dependent phosphorylation. J Biol Chem 279(34):35510-35517.
Ferrer G, et al. (2013) MicroRNA expression in chronic lymphocytic leukemia developing autoimmune hemolytic anemia. Leuk Lymphoma 54(9):2016-2022.
Freedman D A, et al. (1999) Functions of the MDM2 oncoprotein. Cell Mol Life Sci 55:96-107.
Fujiwara T, et al. (1994) Induction of chemosensitivity in human lung cancer cells in vivo by adenovirus-mediated transfer of the wild-type p53 gene. Cancer Res 54(9):2287-2291.
Gadea G, et al. (2004) TNFalpha induces sequential activation of Cdc42- and p38/p53-dependent pathways that antagonistically regulate filopodia formation. J Cell Sci 117(Pt 26):6355-6364.
Gainor J F & Shaw A T (2013) Emerging paradigms in the development of resistance to tyrosine kinase inhibitors in lung cancer. J Clin Oncol 31(31):3987-3996.
Han J et al. (2008) Association of p53 codon 72 polymorphism and MDM2 SNP309 with clinical outcome of advanced nonsmall cell lung cancer. Cancer 113:799-807.
Jackman D M & Johnson B E (2005) Small-cell lung cancer. Lancet 366(9494):1385-1396.
Haupt Y, Maya R, Kazaz A, & Oren M (1997) Mdm2 promotes the rapid degradation of p53. Nature 387(6630):296-299.
He L, et al. (2007) A microRNA component of the p53 tumour suppressor network. Nature 447(7148):1130-1134.
Herbst R S, Heymach J V, & Lippman S M (2008) Lung cancer. N Engl J Med 359(13):1367-1380.
Higashiyama M. et al. (1997) MDM2 gene amplification and expression in non-small-cell lung cancer: immunohistochemical expression of its protein is a favourable prognostic marker in patients without p53 protein accumulation. Br J Cancer 75:1302-1308.
Honda R, Tanaka H, & Yasuda H (1997) Oncoprotein MDM2 is a ubiquitin ligase E3 for tumor suppressor p53. FEBS Lett 420(1):25-27.
Iorio M V, et al. (2005) MicroRNA gene expression deregulation in human breast cancer. Cancer Res 65(16):7065-7070.
Iorio M V & Croce C M (2012) MicroRNA dysregulation in cancer: diagnostics, monitoring and therapeutics. A comprehensive review. EMBO Mol Med 4(3):143-159.
Jemal A, et al. (2011) Global cancer statistics. C A Cancer J Clin 61(2):69-90.
Kosaka N, et al. (2010) Circulating microRNA in body fluid: a new potential biomarker for cancer diagnosis and prognosis. Cancer Sci 101:2087-2092.
Kubbutat M H, Jones S N, & Vousden K H (1997) Regulation of p53 stability by Mdm2. Nature 387(6630):299-303.
Kussie P H, et al. (1996) Structure of the MDM2 oncoprotein bound to the p53 tumor suppressor transactivation domain. Science 274(5289):948-953.
Lagos-Quintana M, Rauhut R, Lendeckel W, & Tuschl T (2001) Identification of novel genes coding for small expressed RNAs. Science 294(5543):853-858.
Lai Z, et al. (2001) Human mdm2 mediates multiple mono-ubiquitination of p53 by a mechanism requiring enzyme isomerization. J Biol Chem 276(33):31357-31367.
Leonard C J, Canman C E, & Kastan M B (1995) The role of p53 in cell-cycle control and apoptosis: implications for cancer. Important Adv Oncol:33-42.
Levine A J (1997) p53, the cellular gatekeeper for growth and division. Cell 88(3):323-331.
Lewis B P, Burge C B, & Bartel D P (2005) Conserved seed pairing, often flanked by adenosines, indicates that thousands of human genes are microRNA targets. Cell 120(1):15-20.
Lynch T J, et al. (2004) Activating mutations in the epidermal growth factor receptor underlying responsiveness of non-small-cell lung cancer to gefitinib. N Engl J Med 350(21):2129-2139.
Ma L, Teruya-Feldstein J, & Weinberg R A (2007) Tumour invasion and metastasis initiated by microRNA-10b in breast cancer. Nature 449(7163):682-688.
Marchetti A, et al. (1995a) mdm2 gene alterations and mdm2 protein expression in breast carcinomas. J Pathol 175(1):31-38.
Marchetti A, et al. (1995b) mdm2 gene amplification and overexpression in non-small cell lung carcinomas with accumulation of the p53 protein in the absence of p53 gene mutations. Diagn Mol Pathol 4(2):93-97.
Moll U M & Petrenko 0 (2003) The MDM2-p53 interaction. Mol Cancer Res 1(14):1001-1008.
Momand J, Zambetti G P, Olson D C, George D, & Levine A J (1992) The mdm-2 oncogene product forms a complex with the p53 protein and inhibits p53-mediated transactivation. Cell 69(7):1237-1245.
Montes de Oca Luna R, Wagner D S, & Lozano G (1995) Rescue of early embryonic lethality in mdm2-deficient mice by deletion of p53. Nature 378(6553):203-206.
Oliner J D, Kinzler K W, Meltzer P S, George D L, & Vogelstein B (1992) Amplification of a gene encoding a p53-associated protein in human sarcomas. Nature 358(6381):80-83.
Pastorino U, et al. (2003) Early lung-cancer detection with spiral CT and positron emission tomography in heavy smokers: 2-year results. Lancet 362(9384):593-597.
Pastorino U, et al. (2012) Annual or biennial CT screening versus observation in heavy smokers: 5-year results of the MILD trial. Eur J Cancer Prev 21(3):308-315.
Peng Y, et al. (2013) Insulin growth factor signaling is regulated by microRNA-486, an underexpressed microRNA in lung cancer. Proc Natl Acad Sci USA 110:15043-15048.
Pizzimenti S, et al. (2009) MicroRNA expression changes during human leukemic HL-60 cell differentiation induced by 4-hydroxynonenal, a product of lipid peroxidation. Free Radic Biol Med 46(2):282-288.
Raponi M, et al. (2009) MicroRNA classifiers for predicting prognosis of squamous cell lung cancer. Cancer Res 69(14):5776-5783.
Riou G, et al. (1995) The p53 and mdm-2 genes in human testicular germ-cell tumors. Mol Carcinog 12(3):124-131.
Rodriguez-Dorantes M, et al. (2014) Prostate cancer detection using a noninvasive method for quantifying miRNAs. Meth Mol Biol 1165:81-87.
Roger L, Gadea G, & Roux P (2006) Control of cell migration: a tumour suppressor function for p53? (Translated from eng) Biol Cell 98(3):141-152.
Socinski M A, et al. (2013) Treatment of stage IV non-small cell lung cancer: Diagnosis and management of lung cancer, 3rd ed: American College of Chest Physicians evidence-based clinical practice guidelines. Chest 143(5 Suppl):e341S-368S.
Soda M, et al. (2007) Identification of the transforming EML4-ALK fusion gene in non-small-cell lung cancer. Nature 448(7153):561-566.
Sozzi G. et al. (2011) MicroRNAs and lung cancer: from markers to targets. Cell Cycle 10:2045-2046.
Sozzi G, et al. (2014) Clinical utility of a plasma-based miRNA signature classifier within computed tomography lung cancer screening: a correlative MILD trial study. J Clin Oncol 32(8):768-773.
Suh S S, et al. (2012) MicroRNAs/TP53 feedback circuitry in glioblastoma multiforme. Proc Natl Acad Sci USA 109(14):5316-5321.
Takamizawa J, et al. (2004) Reduced expression of the let-7 microRNAs in human lung cancers in association with shortened postoperative survival. Cancer Res 64(11):3753-3756.
Tovar C, et al. (2006) Small-molecule MDM2 antagonists reveal aberrant p53 signaling in cancer: implications for therapy. Proc Natl Acad Sci USA 103(6):1888-1893.
Vassilev L T, et al. (2004) In vivo activation of the p53 pathway by small-molecule antagonists of MDM2. Science 303(5659):844-848.
Vogt U, et al. (2002) p53 Gene status in relation to ex vivo chemosensitivity of non-small cell lung cancer. J Cancer Res Clin Oncol 128(3):141-147.
Vousden K H (2000) p53: death star. Cell 103(5):691-694.
Wang Y C, Lin R K, Tan Y H, Chen J T, & Chen C Y (2005) Wild-type p53 overexpression and its correlation with MDM2 and p14ARF alterations: an alternative pathway to non-small-cell lung cancer. J Clin Oncol 23(1):154-164.
Weber J A, et al. (2010) The microRNA spectrum in 12 body fluids. Clin Chem 56:1733-1741.
Weisbart R., et al. 2012. A Cell-Penetrating Bispecific Antibody for Therapeutic Regulation of Intracellular Targets. Mol Cancer Ther 11:2169-2173.
Workman P, et al. (2010) Guidelines for the welfare and use of animals in cancer research. Br J Cancer 102(11):1555-1577.
Wu L & Levine A J (1997) Differential regulation of the p21/WAF-1 and mdm2 genes after high-dose U V irradiation: p53-dependent and p53-independent regulation of the mdm2 gene. Mol Med 3(7):441-451.
Xiao C, et al. (2008) Lymphoproliferative disease and autoimmunity in mice with increased miR-17-92 expression in lymphocytes. Nat Immunol 9(4):405-414.
Xie Q C, et al. (2005) The co-transfection of p16(INK4a) and p14(ARF) genes into human lung cancer cell line A549 and the effects on cell growth and chemosensitivity. Colloids Surf B Biointerfaces 46(3): 188-196.
Xu Y, et al. (2013) Positive expression of p53, c-erbB2 and MRP proteins is correlated with survival rates of NSCLC patients. Mol Clin Oncol 1:487-492.
Yanaihara N, et al. (2006) Unique microRNA molecular profiles in lung cancer diagnosis and prognosis. Cancer Cell 9(3):189-198.
Yokota J & Kohno T (2004) Molecular footprints of human lung cancer progression. Cancer Sci 95(3):197-204.
Youlden D R, Cramb S M, & Baade P D (2008) The International Epidemiology of Lung Cancer: geographical distribution and secular trends. J Thorac Oncol 3(8):819-831.
Zhang J, et al. (2013) Loss of microRNA-143/145 disturbs cellular growth and apoptosis of human epithelial cancers by impairing the MDM2-p53 feedback loop. Oncogene 32(1):61-69.
Zhao Y et al. (2013) Small Molecule Inhibitors of MDM2-p53 and MDMX-p53 Interactions as New Cancer Therapeutics. BioDiscovery 8:4.
Zhu D X, et al. (2012) miR-181a/b significantly enhances drug sensitivity in chronic lymphocytic leukemia cells via targeting multiple anti-apoptosis genes. Carcinogenesis 33(7):1294-1301.
U.S. Pat. No. 7,364,846.
U.S. Pat. No. 7,723,039.
U.S. Pat. No. 7,879,555.
U.S. Pat. No. 8,012,688.
U.S. Pat. No. 8,486,626.
U.S. Pat. No. 8,598,127.
U.S. Pat. No. 8,735,074.
U.S. Patent Publication 2008/0076701.
U.S. Patent Publication 2011/0251098.
U.S. Patent Publication 2012/0329060.
U.S. Patent Publication 2014/0179771.
U.S. Patent Publication 2014/0179763.
PCT Publication No. WO 00/53722.

What is claimed is:

1. A method of treating lung cancer in a patient in need thereof, the method comprising administering to the patient a composition comprising a therapeutically effective amount of a compound that reduces the expression level of E3 ubiquitin-protein ligase MDM2.

2. A method of treating lung cancer in a patient in need thereof, the method comprising administering to the patient a composition comprising a therapeutically effective amount of a miR-660, or a functional variant thereof to treat at least one symptom of lung cancer, wherein the patient in need of treatment (i) expresses miR-660 in a lung tissue sample or biological fluid sample at a level lower than a control level derived from a subject or plurality of subjects that do not have lung cancer, or as compared to a control level derived from a lung cancer patient or plurality thereof, that have been given a favorable prognosis; (ii) expresses MDM2 at a higher level in a lung tissue sample or biological fluid sample as compared to a control level derived from a subject or plurality of subjects that do not have lung cancer, or as compared to a control level derived from a lung cancer patient or plurality thereof that have been given a favorable prognosis; and/or (iii) expresses p53 in a lung tissue sample or a biological fluid sample below a control level derived from a lung tumor tissue sample (or plurality thereof) or a biological fluid sample (or plurality thereof) obtained from a patient that has a favorable lung cancer prognosis; or a control level derived from a healthy subject.

3. The method of claim 1, wherein the compound reduces protein expression of the E3 ubiquitin-protein ligase MDM2.

4. The method of claim 1, wherein the compound reduces expression of the mRNA encoding the E3 ubiquitin-protein ligase MDM2.

5. The method of claim 1, wherein the compound is an antisense RNA, a small interfering RNA (siRNA), short hairpin RNA (shRNA) or a microRNA (miR).

6. The method of claim 1, wherein the compound is a miR.

7. The method of claim 6, wherein the miR is miR-660 or a functional variant thereof.

8. The method of claim 7, wherein miR-660 has the sequence of SEQ ID NO: 2 or SEQ ID NO: 3, or a functional variant thereof.

9. The method of claim 8, wherein the functional variant is at least 80% identical or at least 90% identical in sequence to SEQ ID NO: 2 or SEQ ID NO: 3.

10. The method of claim 7, wherein the miR-660 or functional variant thereof comprises one or more modified nucleotides.

11. The method of claim 7, wherein the miR-660 or functional variant thereof is encoded by a vector.

12. The method of claim 11, wherein the vector is a viral vector.

13. The method of claim 11, wherein the vector is a plasmid based expression vector.

14. The method of claim 12, wherein the viral vector is an adeno-associated virus (AAV) vector.

15. The method of claim 7, wherein the miR-660 or functional variant thereof comprises the sequence ACCCAUU (SEQ ID NO: 4) or ACCCATT (SEQ ID NO: 5).

16. The method of claim 1, wherein the lung cancer is a small cell lung cancer.

17. The method of claim 1, wherein the lung cancer is a non-small cell lung cancer.

18. The method of claim 17, wherein the non-small cell lung cancer is adenocarcinoma.

19. The method of claim 17, wherein the non-small cell lung cancer is squamous cell carcinoma or large-cell lung cancer.

20. The method of claim 1, wherein a lung tumor tissue sample or a biological fluid sample from the patient in need of treatment expresses p53.

21. The method of claim 20, wherein the lung tumor sample or the biological fluid sample from the patient in need of treatment expresses p53 below a control level derived from a lung tumor tissue sample, or a plurality thereof, or a biological fluid sample, or plurality thereof, obtained from a patient that has a favorable lung cancer prognosis; or a control level derived from a lung tissue sample or biological fluid sample obtained from a healthy subject or a plurality of healthy subjects.

22. The method of claim 1, wherein the patient in need of treatment expresses miR-660 in a lung tissue sample or biological fluid sample at a level lower than a control level derived from a subject or plurality of subjects that do not have lung cancer, or as compared to a control level derived from a lung tumor sample or biological fluid sample obtained from a cancer patient or plurality thereof, that have been given a favorable prognosis;

23. The method of claim 1, wherein the patient in need of treatment expresses MDM2 at a higher level in a lung tissue sample or biological fluid sample as compared to a control level derived from a lung tissue sample or biological fluid obtained from a subject or plurality of subjects that do not have lung cancer, or as compared to a control level derived from a lung tissue sample or biological fluid sample obtained from a lung cancer patient or plurality thereof that have been given a favorable prognosis.

24. The method of claim 1, wherein a miR-660 level in a plasma sample from the patient in need of treatment is below a miR-660 level in a plasma sample from a healthy subject or the average miR-660 plasma level in a plurality of healthy subjects.

25. The method of claim 1, wherein a lung tumor tissue sample from the patient in need of treatment expresses MDM2 protein.

26. The method of claim 1, wherein a lung tumor tissue sample from the patient in need of treatment expresses MDM2 mRNA.

27. The method of claim 25, wherein the MDM2 expression level in the lung tumor tissue is greater than an MDM2 level in a noncancerous lung tissue sample from the patient in need of treatment or a healthy subject.

28. The method of claim 1, wherein the patient in need of treatment is identified by a method comprising:
(a) determining the expression ratio of a miRNA pair in a biological sample from the patient;
(b) comparing the expression ratio from step (a) with a cut-off value determined from the average ratio of a plurality of corresponding miRNA pairs from a plurality of control samples;
(c) assigning a positive score for the expression ratio of the miRNA pair in step (a) if the ratio exceeds the cut-off value in step (b);
(d) repeating steps (a) through (c) for additional miRNA pairs until either (1) at least nine miRNA pair expression ratios are assigned a positive score, or (2) after the comparison of the expression ratios of 27 miRNA pairs less than nine miRNA pair expression ratios are assigned a positive score,
wherein the miRNA pairs comprise 106a/140-5p, 106a/142-3p, 126/140-5p, 126/142-3p, 133a/142-3p, 140-5p/17, 142-3p/148a, 142-3p/15b, 142-3p/17, 142-3p/21, 142-3p/221, 142-3p/30b, or 320/660, or the inverse ratios thereof; and
(e) categorizing the presence of a pulmonary tumor as (i) positive if at least nine of the miRNA pair expression ratios are assigned a positive score, or (ii) negative if less than nine miRNA pair expression ratios are assigned a positive score.

29. The method of claim 28, wherein the miRNA pairs further comprise 106a/660, 106a/92a, 126/660, 140-5p/197, 140-5p/28-3p, 142-3p/145, 142-3p/197, 142-3p/28-3p, 17/660, 17/92a, 197/660, 197/92a, 19b/660, or 28-3p/660, or the inverse ratios thereof.
